(19) United States
(12) Patent Application Publication
(10) Pub. No.: US 2024/0190940 A1
(43) Pub. Date: Jun. 13, 2024

(54) HLA CLASS I-RESTRICTED T CELL RECEPTORS AGAINST RAS WITH Q61K MUTATION

(71) Applicant: The United States of America, as represented by the Secretary, Department of Health and Human Serice, Bethesda, MD (US)

(72) Inventors: Paul F. Robbins, Chevy Chase, MD (US); Steven A. Rosenberg, Potomac, MD (US); Gabriel D. Ivey, Washington, DC (US); Almin Lalani, North Bethesda, MD (US)

(73) Assignee: The United States of America, as represented by the Secretary, Department of Health and Human Serice, Bethesda, MD (US)

(21) Appl. No.: 18/286,911

(22) PCT Filed: Apr. 18, 2022
(86) PCT No.: PCT/US2022/025177

§ 371 Date: Oct. 13, 2023

Related U.S. Application Data
(60) Provisional application No. 63/177,570, filed on Apr. 21, 2021.

(51) Int. Cl. C07K 14/725; C12N 15/86

(52) U.S. Cl. CPC C07K 14/7051; C12N 15/86; C12N 2740/10043

(57) ABSTRACT

Disclosed is an isolated or purified T cell receptor (TCR), wherein the TCR has antigenic specificity for a mutated human RAS amino acid sequence with a substitution of glutamine at position 61 with lysine. Related polypeptides and proteins, as well as related nucleic acids, recombinant expression vectors, host cells, populations of cells, and pharmaceutical compositions are also provided. Also disclosed are methods of detecting the presence of cancer in a mammal and methods of treating or preventing cancer in a mammal.

### CROSS-REFERENCE TO RELATED APPLICATION

This patent application claims the benefit of U.S. Provisional Patent Application No. 63/177,570, filed Apr. 21, 2021, which is incorporated by reference in its entirety herein.

### STATEMENT REGARDING FEDERALLY SPONSORED RESEARCH OR DEVELOPMENT

This invention was made with Government support under project number ZIABC010984 by the National Institutes of Health, National Cancer Institute. The Government has certain rights in the invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 57,855 Byte ASCII (Text) file named “759876_ST25.txt,” dated Apr. 18, 2022.

### BACKGROUND OF THE INVENTION

Some cancers may have very limited treatment options, particularly when the cancer becomes metastatic and unresectable. Despite advances in treatments such as, for example, surgery, chemotherapy, and radiation therapy, the prognosis for many cancers, such as, for example, melanoma, pancreatic, colorectal, lung, endometrial, ovarian, and prostate cancers, may be poor. Accordingly, there exists an unmet need for additional treatments for cancer.

### BRIEF SUMMARY OF THE INVENTION

An aspect of the invention provides an isolated or purified T-cell receptor (TCR) having antigenic specificity for a mutated human RAS amino acid sequence with a substitution of glutamine at position 61 with lysine, wherein the mutated human RAS amino acid sequence is a mutated human Kirsten rat sarcoma viral oncogene homolog (KRAS), a mutated human Harvey rat sarcoma viral oncogene homolog (HRAS), or a mutated human Neuroblastoma rat sarcoma viral oncogene homolog (NRAS) amino acid sequence, and wherein position 61 is defined by reference to the wild-type human KRAS, wild-type human HRAS, or wild-type human NRAS protein, respectively.

Another aspect of the invention provides an isolated or purified polypeptide comprising a functional portion of any of the inventive TCRs described herein, wherein the functional portion comprises the amino acid sequences of: (a) all of SEQ ID NOs: 1-3, (b) all of SEQ ID NOs: 4-6, or (c) all of SEQ ID NOs: 1-6.

Still another aspect of the invention provides an isolated or purified protein comprising first and second polypeptide chains, wherein: (a) the first polypeptide chain comprises the amino acid sequences of SEQ ID NOs: 1-3; (b) the a second polypeptide chain comprises the amino acid sequences of SEQ ID NOs: 4-6; or (c) both (a) and (b).

Still another aspect of the invention provides an isolated or purified nucleic acid comprising a nucleotide sequence encoding any of the inventive TCRs, polypeptides, or proteins described herein.

Another aspect of the invention provides an isolated or purified nucleic acid comprising, from 5′ to 3′, a first nucleic acid sequence and a second nucleotide sequence, wherein the first and second nucleotide sequence, respectively, encode the amino sequences of SEQ ID NOs: 7 and 8; 8 and 7; 9 and 10; 10 and 9; 25 and 26; 26 and 25; 27 and 28; 28 and 27; 29 and 30; 30 and 29; 31 and 32; or 32 and 31.

Another aspect of the invention provides a recombinant expression vector comprising any of the inventive nucleic acids described herein.

Another aspect of the invention provides an isolated or purified TCR, polypeptide, or protein encoded by any of the inventive nucleic acids or recombinant expression vectors described herein.

Another aspect of the invention provides an isolated or purified TCR, polypeptide, or protein that results from expression of any of the inventive nucleic acids or recombinant expression vectors described herein in a cell.

Another aspect of the invention provides a method of producing a host cell expressing a TCR that has antigenic specificity for the peptide of ILDTAGKEEY (SEQ ID NO: 37), the method comprising contacting a cell with any of the inventive recombinant expression vectors described herein under conditions that allow introduction of the recombinant expression vector into the cell.

Further aspects of the invention provide an isolated or purified host cell, or an isolated or purified population thereof, comprising any of the inventive nucleic acids or recombinant expression vectors described herein.

Another aspect of the invention provides a method of producing any of the inventive TCRs, polypeptides, or proteins described herein, the method comprising culturing any of the inventive host cells or populations of host cells described herein, so that the TCR, polypeptide, or protein is produced.

Still another aspect of the invention provides a pharmaceutical composition comprising (a) any of the inventive TCRs, polypeptides, proteins, nucleic acids, recombinant expression vectors, host cells, or populations of cells described herein and (b) a pharmaceutically acceptable carrier.

Another embodiment of the invention provides a method of detecting the presence of cancer in mammal, the method comprising: (a) contacting a sample comprising cells of the cancer with any of the inventive TCRs, polypeptides, proteins, nucleic acids, recombinant expression vectors, host cells, populations of cells, or pharmaceutical compositions described herein, thereby forming a complex; and (b) detecting the complex, wherein detection of the complex is indicative of the presence of cancer in the mammal.

Another embodiment of the invention provides a method of inducing an immune response against cancer in a mammal, the method comprising administering to the mammal any of the inventive TCRs, polypeptides, proteins, nucleic acids, recombinant expression vectors, host cells, populations of cells, or pharmaceutical compositions described herein in an amount effective to induce the immune response against cancer in the mammal.

Another embodiment of the invention provides a method of treating or preventing cancer in a mammal, the method comprising administering to the mammal any of the inventive TCRs, polypeptides, proteins, nucleic acids, recombinant expression vectors, host cells, populations of cells, or pharmaceutical compositions described herein in an amount effective to treat or prevent cancer in the mammal.

### BRIEF DESCRIPTION OF THE SEVERAL VIEWS OF THE DRAWING(S)

FIG. 1A is a graph showing IFN-γ secretion (spots/2e4 cells) measured by ELISPOT assay following co-culture of effector cells with target cells. Effector cells were TIL from tumor fragments (numbered F1-F2 and F4-F8) from patient 3795. A combination of TIL from tumor fragments F1, F2, F4, and F5 (“RX1”) served as alternative effector cells. Target cells were DC were (i) independently transfected with mRNA encoding one of 11 TMG constructs (TMG1-TMG11) or (ii) independently pulsed with one of PP1-PP3. Effector cells (i) co-cultured with DC treated with DMSO, (ii) cultured alone (TIL only), or (iii) cultured with DC transfected with a TMG constructing encoding irrelevant mutations (TMGirr) served as negative controls. Effector cells treated with anti-CD3 antibody (OKT3) served as a positive control. The graph legend is shown in FIG. 1E.

FIG. 1B is a graph showing the percentage of cells expressing 4-1BB measured by FACS gated on CD8⁺ cells following co-culture of effector cells with target cells. Effector cells, target cells, and controls are as described for FIG. 1A. The graph legend is shown in FIG. 1E.

FIGS. 1C-1D are graphs showing the percentage of cells expressing 4-1BB (FIG. 1C) or OX40 (FIG. 1D) measured by FACS gated on CD4⁺ cells following co-culture of effector cells with target cells. Effector cells, target cells, and controls are as described for FIG. 1A. The graph legend is shown in FIG. 1E.

FIG. 1E presents the legend for the graphs of FIGS. 1A-1D.

FIG. 1F is a graph showing the percentage of TIL from tumor fragments (numbered F1-F2 and F4-F8) or RX1 from patient 3795 having a CD3⁺, CD4⁺, or CD4⁺ phenotype.

FIG. 2A is a graph showing IFN-γ secretion (spots/2e4 cells) measured by ELISPOT assay following co-culture of effector cells with target cells. Effector cells were TIL from tumor fragments (numbered F1-F2 and F4-F5) from patient 3795. A combination of TIL from tumor fragments F1, F2, F4, and F5 (“RX1”) served as alternative effector cells. Target cells were DC were (i) independently transfected with mRNA encoding one of three TMG constructs (TMG1, TMG2, or TMG6) or (ii) independently pulsed with one of the 32 peptides shown in FIG. 2E. Effector cells (i) co-cultured with DC treated with DMSO, (ii) cultured alone (TIL only), or (iii) cultured with DC transfected with a TMG constructing encoding irrelevant mutations (TMGirr) served as negative controls. Effector cells treated with anti-CD3 antibody (OKT3) served as a positive control. The graph legend is shown in FIG. 2E.

FIG. 2B is a graph showing the percentage of cells expressing 4-1BB measured by FACS gated on CD8⁺ cells following co-culture of effector cells with target cells. Effector cells, target cells, and controls are as described for FIG. 2A. The graph legend is shown in FIG. 2E.

FIGS. 2C-2D are graphs showing the percentage of cells expressing 4-1BB (FIG. 2C) or OX40 (FIG. 2D) measured by FACS gated on CD4⁺ cells following co-culture of effector cells with target cells. Effector cells, target cells, and controls are as described for FIG. 2A. The graph legend is shown in FIG. 2E.

FIG. 2E presents the legend for the graphs of FIGS. 2A-2D.

FIG. 2F is a graph showing the percentage of TIL from tumor fragments (numbered F1-F2 and F4-F5) or RX1 from patient 3795 having a CD3⁺, CD4⁺, or CD4⁺ phenotype.

FIG. 3A is a graph showing the optical density measured following co-culture of TIL from tumor fragment F5 with DC pulsed with various concentrations of mutated NRAS peptides NRAS-0 (circles), NRAS-1 (squares), NRAS-2 (▴), NRAS-3 (▾), or NRAS-R (diamonds).

FIG. 3B present flow cytometry dot plots showing binding of TIL from tumor fragment F5 or TIL used for treatment (RX1) to Q61K RAS tetramer 10-mer.

FIG. 4A shows the percentage of effector cells (of total number of effector cells expressing the TCR of Example 4 (mTCR⁺)) upregulating expression of 4-1BB following co-culture with target cells. Effector cells were T cells sorted for CD4⁺ or CD8⁺ expression and transduced with the retroviral vector of Example 5. Target cells were autologous DCs (Patient 3795) pulsed with the indicated concentrations of the 10-mer ILDTAGKEEY (SEQ ID NO: 37) peptide.

FIG. 4B shows the percentage of effector cells (of total number of effector cells expressing the TCR of Example 4 (mTCR⁺)) upregulating expression of 4-1BB following co-culture with target cells. Effector cells were T cells sorted for CD4⁺ or CD8⁺ expression and transduced with the retroviral vector of Example 5. Target cells were an autologous tumor cell line (3795 TC line) or autologous DCs pulsed with 1 or 10 μg/ml of wild-type (WT) ILDTAGQEEY (SEQ ID NO: 44) peptide or 1 μg/ml of 10-mer ILDTAGREEY (SEQ ID NO: 41) (NRAS-R). DCs treated with DMSO and T cells cultured alone served as controls.

### DETAILED DESCRIPTION OF THE INVENTION

RAS family proteins belong to the large family of small GTPases. Without being bound to a particular theory or mechanism, it is believed that, when mutated, RAS proteins may be involved in signal transduction early in the oncogenesis of many human cancers. A single amino acid substitution may activate the protein. The mutated RAS protein product may be constitutively activated. Mutated RAS proteins may be expressed in any of a variety of human cancers such as, for example, melanoma, pancreatic (e.g., pancreatic carcinoma), colorectal, lung (e.g., lung adenocarcinoma), endometrial, ovarian (e.g., epithelial ovarian cancer), and prostate cancers. The human RAS family proteins include KRAS, HRAS, and NRAS.

KRAS is also referred to as GTPase KRas, V-Ki-Ras2 Kirsten rat sarcoma viral oncogene, or KRAS2. There are two transcript variants of KRAS: KRAS variant A and KRAS variant B. Wild-type (WT) KRAS variant A has the amino acid sequence of SEQ ID NO: 11. WT KRAS variant B has the amino acid sequence of SEQ ID NO: 12. Hereinafter, references to “KRAS” (mutated or unmutated (WT)) refer to both variant A and variant B. unless specified otherwise. When activated, mutated KRAS binds to guanosine-5′-triphosphate (GTP) and converts GTP to guanosine 5′-diphosphate (GDP).

HRAS is another member of the RAS protein family. HRAS is also referred to as Harvey Rat Sarcoma Viral Oncoprotein, V-Ha-Ras Harvey Rat Sarcoma Viral Oncogene Homolog, or Ras Family Small GTP Binding Protein H-Ras. WT HRAS has the amino acid sequence of SEQ ID NO: 13.

NRAS is still another member of the RAS protein family. NRAS is also referred to as GTPase NRas, V-Ras Neuroblastoma RAS Viral Oncogene Homolog, or NRAS1. WT NRAS has the amino acid sequence of SEQ ID NO: 14.

An aspect of the invention provides an isolated or purified TCR, wherein the TCR has antigenic specificity for a mutated human RAS amino acid sequence with a substitution of glutamine at position 61 with lysine, wherein the mutated human RAS amino acid sequence is a mutated human KRAS, a mutated human HRAS, or a mutated human NRAS amino acid sequence, and wherein position 61 is defined by reference to the WT human KRAS, WT human HRAS, or WT human NRAS protein, respectively. Hereinafter, references to a “TCR” also refer to functional portions and functional variants of the TCR, unless specified otherwise.

The mutated human RAS amino acid sequence may be a mutated human KRAS amino acid sequence, a mutated human HRAS amino acid sequence, or a mutated human NRAS amino acid sequence. The amino acid sequences of WT human KRAS, NRAS, and HRAS protein each have a length of 188 or 189 amino acid residues and have a high degree of identity to one another. For example, the amino acid sequence of the WT human NRAS protein is 86.8% identical to that of the WT human KRAS protein. Amino acid residues 1-86 of the WT human NRAS protein and the WT human KRAS protein are 100% identical. The amino acid sequence of the WT human HRAS protein is 86.3% identical to that of the WT human KRAS protein. Amino acid residues 1-94 of the WT human HRAS protein and the WT human KRAS protein are 100% identical. Hereinafter, references to “RAS” (mutated or unmutated (WT)) collectively refer to KRAS, HRAS, and NRAS, unless specified otherwise.

In an aspect of the invention, the mutated human RAS amino acid sequence comprises a human RAS amino acid sequence with a substitution of glutamine at position 61 with lysine, wherein position 61 is defined by reference to the corresponding WT RAS protein. The WT RAS protein may be any one of WT KRAS protein (SEQ ID NO: 11 or 12), WT HRAS protein (SEQ ID NO: 13), or WT NRAS protein (SEQ ID NO: 14) because, as explained above, amino acid residues 1-86 of the WT human NRAS protein and the WT human KRAS protein are 100% identical, and amino acid residues 1-94 of the WT human HRAS protein and the WT human KRAS protein are 100% identical. Accordingly, the amino acid residue at position 61 of each of WT KRAS, WT HRAS, and WT NRAS protein is the same, namely, glutamine.

The mutated human RAS amino acid sequence has a substitution of glutamine at position 61 with lysine. In this regard, aspects of the invention provide TCRs with antigenic specificity for any human RAS protein, polypeptide or peptide amino acid sequence with a Q61K mutation.

Mutations and substitutions of RAS are defined herein by reference to the amino acid sequence of the corresponding WT RAS protein. Thus, mutations and substitutions of RAS are described herein by reference to the amino acid residue present at a particular position in WT RAS protein (namely, position 61), followed by the position number, followed by the amino acid residue with which that residue has been replaced in the particular mutation or substitution under discussion. A RAS amino acid sequence (e.g., a RAS peptide) may comprise fewer than all of the amino acid residues of the full-length, WT RAS protein. Accordingly, position 61 is defined herein by reference to the WT full-length RAS protein (namely, any one of SEQ ID NOs: 11-14) with the understanding that the actual position of the corresponding residue in a particular example of a RAS amino acid sequence may be different. When the positions are as defined by any one of SEQ ID NOs: 11-14, the term “Q61” refers to the glutamine normally present at position 61 of any one of SEQ ID NOs: 11-14, and “Q61K” indicates that the glutamine normally present at position 61 of any one of SEQ ID NOs: 11-14 is replaced by lysine. For example, when a particular example of a RAS amino acid sequence is, e.g., ILDTAGQEEY (SEQ ID NO: 44) (an exemplary WT NRAS peptide corresponding to contiguous amino acid residues 55 to 64 of SEQ ID NO: 14), “Q61K” refers to a substitution of the underlined glutamine in SEQ ID NO: 44 with lysine, even though the actual position of the underlined glutamine in SEQ ID NO: 44 is 7. Human RAS amino acid sequences with the Q61K mutation are hereinafter referred to as “Q61K RAS.”

Examples of full-length RAS proteins with the Q61K mutation are set forth in Table 1 below.

TABLE 1

| Mutated Full-Length RAS Protein | SEQ ID NO: |
|---|---|
| Q61K KRAS variant A | 15 |
| Q61K KRAS variant B | 16 |
| Q61K HRAS | 17 |
| Q61K NRAS | 18 |

In an aspect of the invention, the TCR has antigenic specificity for a RAS peptide with the Q61K mutation described above, wherein the Q61K RAS peptide has any length. In an aspect of the invention, the Q61K RAS peptide has any length suitable for binding to any of the HLA Class I molecules described herein. For example, the TCR may have antigenic specificity for a RAS peptide with the Q61K mutation, the RAS peptide having a length of about 9 to about 10 amino acid residues. The Q61K RAS peptide may comprise any contiguous amino acid residues of mutated RAS protein which include the Q61K mutation. An example of a specific peptide with the Q61K mutation, which may be recognized by the inventive TCRs, is ILDTAGKEEY (SEQ ID NO: 37). In an aspect of the invention, the TCR has antigenic specificity for the mutated human RAS amino acid sequence of SEQ ID NO: 37. In an aspect of the invention, the TCR does not have antigenic specificity for the wild-type human RAS amino acid sequence of ILDTAGQEEY (SEQ ID NO: 44). In an aspect of the invention, the TCR does not have antigenic specificity for the Q61R RAS amino acid sequence of ILDTAGREEY (SEQ ID NO: 41).

In an aspect of the invention, the inventive TCRs are able to recognize Q61K RAS presented by an HLA Class I molecule. In this regard, the TCR may elicit an immune response upon binding to Q61K RAS presented by an HLA Class I molecule. The inventive TCRs may bind to the HLA Class I molecule in addition to Q61K RAS.

In an embodiment of the invention, the HLA Class I molecule is an HLA-A molecule. The HLA-A molecule is a heterodimer of an α chain and β2 microglobulin. The HLA-A α chain may be encoded by an HLA-A gene. β2 microglobulin binds non-covalently to the alpha1, alpha2 and alpha3 domains of the alpha chain to build the HLA-A complex. The HLA-A molecule may be any HLA-A molecule. In an embodiment of the invention, the HLA Class I molecule is an HLA-A1 molecule. The HLA-A1 molecule may be any HLA-A1 molecule. Examples of HLA-A1 molecules may include, but are not limited to, those encoded by the HLA-A*01:01, HLA-A*01:02, or HLA-A*01:03 allele. Preferably, the HLA Class I molecule is encoded by the HLA-A*01:01 allele.

The TCRs of the invention may provide any one or more of a variety of advantages, including when expressed by cells used for adoptive cell transfer. Q61K RAS is expressed by cancer cells and is not expressed by normal, noncancerous cells. Without being bound to a particular theory or mechanism, it is believed that the inventive TCRs advantageously target the destruction of cancer cells while minimizing or eliminating the destruction of normal, non-cancerous cells, thereby reducing, for example, by minimizing or eliminating, toxicity. Moreover, because the Q61K mutation is likely to occur in the early stages of tumorigenesis, the Q61K RAS mutation may be expressed on substantially all of a patient's cancer cells. The inventive TCRs may, advantageously, successfully treat or prevent Q61K RAS-positive cancers that do not respond to other types of treatment such as, for example, chemotherapy, surgery, or radiation. Additionally, the inventive TCRs may provide highly avid recognition of Q61K RAS, which may provide the ability to recognize unmanipulated tumor cells (e.g., tumor cells that have not been treated with interferon (IFN)-γ, transfected with a vector encoding one or both of Q61K RAS and and any of the HLA Class I molecules described herein, pulsed with a Q61K RAS peptide, or a combination thereof). The frequency of NRAS mutations in malignant melanomas is about 13 to about 25% (Catalogue Of Somatic Mutations In Cancer (COSMIC)). The frequency of Q61K mutations among NRAS-mutated malignant melanomas is about 34% (COSMIC). Moreover, the percentage of the U.S. population expressing the HLA-A*01:01 allele is about 8 to about 15% (allelefrequencies.net). Accordingly, the inventive TCRs may increase the number of immunotherapy-eligible cancer patients to include those patients that express the HLA-A*01:01 allele who may not be eligible for immunotherapy using TCRs that recognize Q61K RAS presented by other MHC molecules. Moreover, the inventive TCRs, polypeptides and proteins comprise human CDR and variable region amino acid sequences, which may reduce the risk of rejection by the human immune system as compared to, e.g., TCRs, polypeptides and proteins comprising mouse CDR and variable region amino acid sequences.

The phrase “antigenic specificity,” as used herein, means that the TCR can specifically bind to and immunologically recognize Q61K RAS with high avidity. For example, a TCR may be considered to have “antigenic specificity” for Q61K RAS if about 1×10⁴ to about 1×10⁵ T cells expressing the TCR secrete at least about 200 pg/mL or more (e.g., 200 pg/mL or more, 300 pg/mL or more, 400 pg/mL or more, 500 pg/mL or more, 600 pg/mL or more, 700 pg/mL or more, 1000 pg/mL or more, 5,000 pg/mL or more, 7,000 pg/mL or more, 10,000 pg/mL or more, 20,000 pg/mL or more, or a range defined by any two of the foregoing values) of IFN-γ upon co-culture with (a) antigen-negative, HLA Class I molecule positive target cells pulsed with a low concentration of Q61K RAS peptide (e.g., about 0.05 ng/ml to about 10 ng/ml, 1 ng/mL, 2 ng/mL, 5 ng/ml, 8 ng/ml, 10 ng/ml, or a range defined by any two of the foregoing values) or (b) antigen-negative, HLA Class I molecule positive target cells into which a nucleotide sequence encoding Q61K RAS has been introduced such that the target cell expresses Q61K RAS. Cells expressing the inventive TCRs may also secrete IFN-γ upon co-culture with antigen-negative, HLA Class I molecule positive target cells pulsed with higher concentrations of Q61K RAS peptide. The HLA Class I molecule may be any of the HLA Class I molecules described herein.

Alternatively or additionally, a TCR may be considered to have “antigenic specificity” for Q61K RAS if T cells expressing the TCR secrete at least twice (e.g., five times) as much IFN-γ upon co-culture with (a) antigen-negative, HLA Class I molecule positive target cells pulsed with a low concentration of Q61K RAS peptide or (b) antigen-negative, HLA Class I molecule positive target cells into which a nucleotide sequence encoding Q61K RAS has been introduced such that the target cell expresses Q61K RAS as compared to the amount of IFN-γ expressed by a negative control. The negative control may be, for example, (i) T cells expressing the TCR, co-cultured with (a) antigen-negative, HLA Class I molecule positive target cells pulsed with the same concentration of an irrelevant peptide (e.g., some other peptide with a different sequence from the Q61K RAS peptide) or (b) antigen-negative, HLA Class I molecule positive target cells into which a nucleotide sequence encoding an irrelevant peptide has been introduced such that the target cell expresses the irrelevant peptide, or (ii) untransduced T cells (e.g., derived from PBMC, which do not express the TCR) co-cultured with (a) antigen-negative, HLA Class I molecule positive target cells pulsed with the same concentration of Q61K RAS peptide or (b) antigen-negative, HLA Class I molecule positive target cells into which a nucleotide sequence encoding Q61K RAS has been introduced such that the target cell expresses Q61K RAS. The HLA Class I molecule expressed by the target cells of the negative control would be the same HLA Class I molecule expressed by the target cells that are co-cultured with the T cells being tested. The HLA Class I molecule may be any of the HLA Class I molecules described herein. IFN-γ secretion may be measured by methods known in the art such as, for example, enzyme-linked immunosorbent assay (ELISA).

Alternatively or additionally, a TCR may be considered to have “antigenic specificity” for Q61K RAS if at least twice (e.g., five times) as many of the numbers of T cells expressing the TCR secrete IFN-γ upon co-culture with (a) antigen-negative, HLA Class I molecule positive target cells pulsed with a low concentration of Q61K RAS peptide or (b) antigen-negative, HLA Class I molecule positive target cells into which a nucleotide sequence encoding Q61K RAS has been introduced such that the target cell expresses Q61K RAS as compared to the numbers of negative control T cells that secrete IFN-γ. The HLA Class I molecule, concentration of peptide, and the negative control may be as described herein with respect to other aspects of the invention. The numbers of cells secreting IFN-γ may be measured by methods known in the art such as, for example, ELISPOT.

Alternatively or additionally, a TCR may be considered to have “antigenic specificity” for Q61K RAS if T cells expressing the TCR upregulate expression of one or more T-cell activation markers as measured by, for example, flow cytometry after stimulation with target cells expressing Q61K RAS. Examples of T-cell activation markers include 4-1BB, OX40, CD107a, CD69, and cytokines that are upregulated upon antigen stimulation (e.g., tumor necrosis factor (TNF), interleukin (IL)-2, etc.).

An aspect of the invention provides a TCR comprising two polypeptides (i.e., polypeptide chains), such as an alpha (α) chain of a TCR, a beta (β) chain of a TCR, a gamma (γ) chain of a TCR, a delta (δ) chain of a TCR, or a combination thereof. The polypeptides of the inventive TCR can comprise any amino acid sequence, provided that the TCR has antigenic specificity for Q61K RAS. In some aspects, the TCR is non-naturally occurring.

In an aspect of the invention, the TCR comprises two polypeptide chains, each of which comprises a variable region comprising a complementarity determining region (CDR)1, a CDR2, and a CDR3 of a TCR. In an aspect of the invention, the TCR comprises a first polypeptide chain comprising a CDR1 comprising the amino acid sequence of SEQ ID NO: 1 (CDR1 of α chain), a CDR2 comprising the amino acid sequence of SEQ ID NO: 2 (CDR2 of α chain), and a CDR3 comprising the amino acid sequence of SEQ ID NO: 3 (CDR3 of α chain), and a second polypeptide chain comprising a CDR1 comprising the amino acid sequence of SEQ ID NO: 4 (CDR1 of β chain), a CDR2 comprising the amino acid sequence of SEQ ID NO: 5 (CDR2 of β chain), and a CDR3 comprising the amino acid sequence of SEQ ID NO: 6 (CDR3 of β chain). In this regard, the inventive TCR can comprise any one or more of the amino acid sequences selected from the group consisting of SEQ ID NOs: 1-6. In an aspect of the invention, the TCR comprises the amino acid sequences of: (a) all of SEQ ID NOs: 1-3, (b) all of SEQ ID NOs: 4-6, or (c) all of SEQ ID NOs: 1-6. In an especially preferred aspect, the TCR comprises the amino acid sequences of all of SEQ ID NOs: 1-6.

In an aspect of the invention, the TCR comprises an amino acid sequence of a variable region of a TCR comprising the CDRs set forth above. In this regard, the TCR can comprise the amino acid sequence of: (1) SEQ ID NO: 7 (predicted sequence of variable region of α chain without N-terminal signal peptide); (2) SEQ ID NO: 8 (predicted sequence of variable region of β chain without N-terminal signal peptide); (3) SEQ ID NO: 9 (variable region of α chain with N-terminal signal peptide); (4) SEQ ID NO: 10 (variable region of β chain with N-terminal signal peptide); (5) both of SEQ ID NOs: 7 and 8; or (6) both of SEQ ID NOs: 9 and 10. Preferably, the TCR comprises the amino acid sequences of (i) both of SEQ ID NOs: 7 and 8 or (ii) both of SEQ ID NOs: 9 and 10.

The inventive TCRs may further comprise an α chain constant region and a β chain constant region. The constant region may be derived from any suitable species such as, e.g., human or mouse. In an aspect of the invention, the TCRs further comprise murine α and β chain constant regions or human α and β chain constant regions. As used herein, the term “murine” or “human,” when referring to a TCR or any component of a TCR described herein (e.g., CDR, variable region, constant region, α chain, and/or β chain), means a TCR (or component thereof) which is derived from a mouse or a human, respectively, i.e., a TCR (or component thereof) that originated from or was, at one time, expressed by a mouse T cell or a human T cell, respectively.

An aspect of the invention provides a chimeric TCR comprising a human variable region and a murine constant region, wherein the TCR has antigenic specificity for a mutated human RAS amino acid sequence with a substitution of glutamine at position 61 with lysine. The murine constant region may provide any one or more advantages. For example, the murine constant region may diminish mispairing of the inventive TCR with the endogenous TCRs of the host cell into which the inventive TCR is introduced. Alternatively or additionally, the murine constant region may increase expression of the inventive TCR as compared to the same TCR with a human constant region. The chimeric TCR may comprise the amino acid sequence of SEQ ID NO: 23 (WT murine α chain constant region), SEQ ID NO: 24 (WT murine β chain constant region), or both SEQ ID NOs: 23 and 24. Preferably, the inventive TCR comprises the amino acid sequences of both of SEQ ID NOs: 23 and 24. The chimeric TCR may comprise any of the murine constant regions described herein in combination with any of the CDR regions as described herein with respect to other aspects of the invention. In this regard, the TCR may comprise the amino acid sequences of: (a) all of SEQ ID NOs: 1-3 and 23, (b) all of SEQ ID NOs: 4-6 and 24, or (c) all of SEQ ID NOs: 1-6 and 23-24.

In another aspect of the invention, the chimeric TCR may comprise any of the murine constant regions described herein in combination with any of the variable regions described herein with respect to other aspects of the invention. In this regard, the TCR may comprise the amino acid sequences of: (1) both of SEQ ID NOs: 7 and 23, (2) both of SEQ ID NOs: 8 and 24, (3) both of SEQ ID NOs: 9 and 23, (4) both of SEQ ID NOs: 10 and 24, (5) all of SEQ ID NOs: 7-8 and 23-24, or (6) all of SEQ ID NOs: 9-10 and 23-24.

In an aspect of the invention, the TCR comprises a substituted constant region. In this regard, the TCR may comprise the amino acid sequence of any of the TCRs described herein with one, two, three, or four amino acid substitution(s) in the constant region of one or both of the α and β chain. Preferably, the TCR comprises a murine constant region with one, two, three, or four amino acid substitution(s) in the murine constant region of one or both of the α and β chains. In an especially preferred aspect, the TCR comprises a murine constant region with one, two, three, or four amino acid substitution(s) in the murine constant region of the α chain and one amino acid substitution in the murine constant region of the β chain. In some aspects, the TCRs comprising the substituted constant region advantageously provide one or more of increased recognition of Q61K RAS⁺ targets, increased expression by a host cell, diminished mispairing with endogenous TCRs, and increased anti-tumor activity as compared to the parent TCR comprising an unsubstituted (wild-type) constant region. In general, the substituted amino acid sequences of the murine constant regions of the TCR α and β chains, SEQ ID NOs: 19 and 20, respectively, correspond with all or portions of the unsubstituted murine constant region amino acid sequences SEQ ID NOs: 23 and 24, respectively, with SEQ ID NO: 19 having one, two, three, or four amino acid substitution(s) when compared to SEQ ID NO: 23 and SEQ ID NO: 20 having one amino acid substitution when compared to SEQ ID NO: 24. In this regard, an aspect of the invention provides a TCR comprising the amino acid sequences of (a) SEQ ID NO: 19 (constant region of α chain). wherein (i) X at position 48 is Thr or Cys; (ii) X at position 112 is Ser, Ala, Val, Leu, Ile, Pro, Phe, Met, or Trp; (iii) X at position 114 is Met, Ala, Val, Leu, Ile, Pro, Phe, or Trp; and (iv) X at position 115 is Gly, Ala, Val, Leu, Ile, Pro, Phe, Met, or Trp; (b) SEQ ID NO: 20 (constant region of β chain), wherein X at position 57 is Ser or Cys; or (c) both of SEQ ID NOs: 19 and 20. In an aspect of the invention, the TCR comprising SEQ ID NO: 19 does not comprise SEQ ID NO: 23 (unsubstituted murine constant region of α chain). In an aspect of the invention, the TCR comprising SEQ ID NO: 20 does not comprise SEQ ID NO: 24 (unsubstituted murine constant region of β chain).

In an aspect of the invention, the TCR comprises an α chain comprising a variable region and a constant region and a β chain comprising a variable region and a constant region. In this regard, the TCR may comprise (a) the amino acid sequence of SEQ ID NO: 25 (α chain with N-terminal signal peptide), wherein: (i) X at position 185 of SEQ ID NO: 25 is Thr or Cys; (ii) X at position 249 of SEQ ID NO: 25 is Ser, Ala, Val, Leu, Ile, Pro, Phe, Met, or Trp; (iii) X at position 251 of SEQ ID NO: 25 is Met, Ala, Val, Leu, Ile, Pro, Phe, or Trp; and (iv) X at position 252 of SEQ ID NO: 25 is Gly, Ala, Val, Leu, Ile, Pro, Phe, Met, or Trp; (b) the amino acid sequence of SEQ ID NO: 26 (β chain with N-terminal signal peptide), wherein X at position 186 of SEQ ID NO: 26 is Ser or Cys; (c) the amino acid sequences of both of SEQ ID NOs: 25 and 26; (d) the amino acid sequence of SEQ ID NO: 27 (predicted sequence of α chain without N-terminal signal peptide), wherein: (i) X at position 165 of SEQ ID NO: 27 is Thr or Cys; (ii) X at position 229 of SEQ ID NO: 27 is Ser, Ala, Val, Leu, Ile, Pro, Phe, Met, or Trp; (iii) X at position 231 of SEQ ID NO: 27 is Met, Ala, Val, Leu, Ile, Pro, Phe, or Trp; and (iv) X at position 232 of SEQ ID NO: 27 is Gly, Ala, Val, Leu, Ile, Pro, Phe, Met, or Trp; (e) the amino acid sequence of SEQ ID NO: 28 (predicted sequence of β chain without N-terminal signal peptide), wherein X at position 167 of SEQ ID NO: 28 is Ser or Cys; (f) the amino acid sequences of both of SEQ ID NOs: 27 and 28; (g) the amino acid sequence of SEQ ID NO: 29 (α chain of cysteine-substituted, LVL-modified TCR with N-terminal signal peptide); (h) the amino acid sequence of SEQ ID NO: 30 (β chain of cysteine-substituted, LVL-modified TCR with N-terminal signal peptide); (i) the amino acid sequence of SEQ ID NO: 31 (predicted sequence of α chain of cysteine-substituted, LVL-modified TCR without N-terminal signal peptide); (j) the amino acid sequence of SEQ ID NO: 32 (predicted sequence of β chain of cysteine-substituted, LVL-modified TCR without N-terminal signal peptide); (k) the amino acid sequences of both of SEQ ID NOs: 29 and 30; or (l) the amino acid sequences of both of SEQ ID NOs: 31 and 32.

In an aspect of the invention, the substituted constant region includes cysteine substitutions in the constant region of one or both of the α and β chains to provide a cysteine-substituted TCR. Opposing cysteines in the α and the β chains provide a disulfide bond that links the constant regions of the α and the β chains of the substituted TCR to one another and which is not present in a TCR comprising the unsubstituted murine constant regions. In this regard, the TCR may be a cysteine-substituted TCR in which one or both of the native Thr at position 48 (Thr48) of SEQ ID NO: 23 and the native Ser at position 57 (Ser57) of SEQ ID NO: 24 may be substituted with Cys. Preferably, both of the native Thr48 of SEQ ID NO: 23 and the native Ser57 of SEQ ID NO: 24 are substituted with Cys. Examples of cysteine-substituted TCR constant regions sequences are set forth in Table 2. In an aspect of the invention, the cysteine-substituted TCR comprises (i) SEQ ID NO: 19, (ii) SEQ ID NO: 20, or (iii) both of SEQ ID NOs: 19 and 20, wherein both of SEQ ID NOs: 19 and 20 are as defined in Table 2. The cysteine-substituted TCRs of the invention may include the substituted constant region in addition to any of the CDRs or variable regions described herein.

In an aspect of the invention, the cysteine-substituted, chimeric TCR comprises a full length α chain and a full-length β chain. Examples of cysteine-substituted, chimeric TCR α chain and β chain sequences are set forth in Table 2. In an aspect of the invention, the TCR comprises: (1) SEQ ID NO: 19, (2) SEQ ID NO: 20, (3) SEQ ID NO: 25, (4) SEQ ID NO: 26, (5) SEQ ID NO: 27, (6) SEQ ID NO: 28, (7) both of SEQ ID NOs: 19 and 20, (8) both of SEQ ID NOs: 25 and 26, or (9) both of SEQ ID NOs: 27 and 28, wherein all of SEQ ID NOs: 19-20 and 25-28 are as defined in Table 2.

TABLE 2

| SEQ ID NO: | Definitions of “X” |
|---|---|
| SEQ ID NO: 19 | X at position 48 is Cys, |
| (constant region α chain) | X at position 112 is Ser, |
| | X at position 114 is Met, and |
| | X at position 115 is Gly. |
| SEQ ID NO: 20 | X at position 57 is Cys |
| (constant region β chain) | |
| SEQ ID NO: 25 | X at position 185 is Cys, |
| (α chain with N-terminal | X at position 249 is Ser, |
| signal peptide) | X at position 251 is Met, and |
| | X at position 252 is Gly. |
| SEQ ID NO: 26 | X at position 186 is Cys |
| (β chain with N-terminal | |
| signal peptide) | |
| SEQ ID NO: 27 | X at position 165 is Cys, |
| (α chain predicted sequence | X at position 229 is Ser, |
| without N-terminal signal peptide) | X at position 231 is Met, and |
| | X at position 232 is Gly. |
| SEQ ID NO: 28 | X at position 167 is Cys |
| (β chain predicted sequence | |
| without N-terminal signal peptide) | |

In an aspect of the invention, the substituted amino acid sequence includes substitutions of one, two, or three amino acids in the transmembrane (TM) domain of the constant region of the α chain with a hydrophobic amino acid to provide a hydrophobic amino acid-substituted TCR (also referred to herein as an “LVL-modified TCR”). The hydrophobic amino acid substitution(s) in the TM domain of the TCR may increase the hydrophobicity of the TM domain of the TCR as compared to a TCR that lacks the hydrophobic amino acid substitution(s) in the TM domain. In this regard, the TCR is an LVL-modified TCR in which one, two, or three of the native Ser112, Met114, and Gly115 of SEQ ID NO: 23 may, independently, be substituted with Ala, Val, Leu, Ile, Pro, Phe, Met, or Trp; preferably with Leu, Ile, or Val. Preferably, all three of the native Ser112, Met114, and Gly 115 of SEQ ID NO: 23 may, independently, be substituted with Ala, Val, Leu, Ile, Pro, Phe, Met, or Trp; preferably with Leu, Ile, or Val. In an aspect of the invention, the LVL-modified TCR comprises (i) SEQ ID NO: 19, (ii) SEQ ID NO: 20, or (iii) both of SEQ ID NOs: 19 and 20, wherein both of SEQ ID NOs: 19 and 20 are as defined in Table 3. The LVL-modified TCRs of the invention may include the substituted constant region in addition to any of the CDRs or variable regions described herein.

In an aspect of the invention, the LVL-modified TCR comprises a full length α chain and a full-length β chain. Examples of LVL-modified TCR α chain and β chain sequences are set forth in Table 3. In an aspect of the invention, the TCR comprises: (1) SEQ ID NO: 19, (2) SEQ ID NO: 20, (3) SEQ ID NO: 25, (4) SEQ ID NO: 26, (5) SEQ ID NO: 27, (6) SEQ ID NO: 28, (7) both of SEQ ID NOs: 19 and 20, (8) both of SEQ ID NOs: 25 and 26, or (9) both of SEQ ID NOs: 27 and 28, wherein all of SEQ ID NOs: 19-20 and 25-28 are as defined in Table 3.

TABLE 3

| SEQ ID NO: | Definitions of “X” |
|---|---|
| SEQ ID NO: 19 | X at position 48 is Thr; |
| (constant region α | X at position 112 is Ser, Ala, Val, Leu, Ile, Pro, Phe, Met, or Trp; |
| chain) | preferably wherein X at position 112 is Leu, Ile, or Val; |
| | especially preferably wherein X at position 112 is Leu; |
| | X at position 114 is Met, Ala, Val, Leu, Ile, Pro, Phe, or Trp; |
| | preferably wherein X at position 114 is Leu, Ile, or Val; |
| | especially preferably wherein X at position 114 is Ile; and |
| | X at position 115 is Gly, Ala, Val, Leu, Ile, Pro, Phe, Met, or Trp; |
| | preferably wherein X at position 115 is Leu, Ile, or Val; |
| | especially preferably wherein X at position 115 is Val; |
| | wherein SEQ ID NO: 19 does not comprise SEQ ID NO: 23 |
| | (unsubstituted α chain constant region) |
| SEQ ID NO: 20 | X at position 57 is Ser |
| (constant region β | |
| chain) | |
| SEQ ID NO: 25 (α | X at position 185 is Thr; |
| chain) (with N- | X at position 249 is Ser, Ala, Val, Leu, Ile, Pro, Phe, Met, or Trp; |
| terminal signal | preferably wherein X at position 249 is Leu, Ile, or Val; |
| peptide) | especially preferably wherein X at position 249 is Leu; |
| | X at position 251 is Met, Ala, Val, Leu, Ile, Pro, Phe, or Trp; |
| | preferably wherein X at position 251 is Leu, Ile, or Val; |
| | especially preferably wherein X at position 251 is Ile; and |
| | X at position 252 is Gly, Ala, Val, Leu, Ile, Pro, Phe, Met, or Trp; |
| | preferably wherein X at position 252 is Leu, Ile, or Val; |
| | especially preferably wherein X at position 252 is Val, |
| | wherein SEQ ID NO: 25 does not comprise SEQ ID NO: 23 |
| | (unsubstituted α chain constant region) |
| SEQ ID NO: 26 (β | X at position 186 is Ser |
| chain) (with N- | |
| terminal signal | |
| peptide) | |
| SEQ ID NO: 27 (α | X at position 165 is Thr; |
| chain) (predicted | X at position 229 is Ser, Ala, Val, Leu, Ile, Pro, Phe, Met, or Trp; |
| sequence without N- | preferably wherein X at position 229 is Leu, Ile, or Val; |
| terminal signal | especially preferably wherein X at position 229 is Leu; |
| peptide) | X at position 231 is Met, Ala, Val, Leu, Ile, Pro, Phe, or Trp; |
| | preferably wherein X at position 231 is Leu, Ile, or Val; |
| | especially preferably wherein X at position 231 is Ile; and |
| | X at position 232 is Gly, Ala, Val, Leu, Ile, Pro, Phe, Met, or Trp; |
| | preferably wherein X at position 232 is Leu, Ile, or Val; |
| | especially preferably wherein X at position 232 is Val, |
| | wherein SEQ ID NO: 27 does not comprise SEQ ID NO: 23 |
| | (unsubstituted α chain constant region) |
| SEQ ID NO: 28 (β | X at position 167 is Ser |
| chain) (predicted | |
| sequence without N- | |
| terminal signal | |
| peptide) | |

In an aspect of the invention, the substituted amino acid sequence includes the cysteine substitutions in the constant region of one or both of the α and β chains in combination with the substitution(s) of one, two, or three amino acids in the transmembrane (TM) domain of the constant region of the α chain with a hydrophobic amino acid (also referred to herein as “cysteine-substituted, LVL-modified TCR”). In this regard, the TCR is a cysteine-substituted, LVL-modified, chimeric TCR in which the native Thr48 of SEQ ID NO: 23 is substituted with Cys; one, two, or three of the native Ser112, Met114, and Gly 115 of SEQ ID NO: 23 are, independently, substituted with Ala, Val, Leu, Ile, Pro, Phe, Met, or Trp; preferably with Leu, Ile, or Val; and the native Ser57 of SEQ ID NO: 24 is substituted with Cys. Preferably, all three of the native Ser112, Met114, and Gly 115 of SEQ ID NO: 23 may, independently, be substituted with Ala, Val, Leu, Ile, Pro, Phe, Met, or Trp; preferably with Leu, Ile, or Val. In an aspect of the invention, the cysteine-substituted, LVL-modified TCR comprises (i) SEQ ID NO: 19, (ii) SEQ ID NO: 20, or (iii) both of SEQ ID NOs: 19 and 20, wherein both of SEQ ID NOs: 19 and 20 are as defined in Table 4. The cysteine-substituted, LVL-modified TCRs of the invention may include the substituted constant region in addition to any of the CDRs or variable regions described herein.

In an aspect, the cysteine-substituted, LVL-modified TCR comprises a full-length α chain and a full-length β chain. Examples of cysteine-substituted, LVL-modified TCR α chain and β chain sequences are set forth in Tables 4 and 12. In an aspect of the invention, the TCR comprises: (1) SEQ ID NO: 19, (2) SEQ ID NO: 20, (3) SEQ ID NO: 25, (4) SEQ ID NO: 26, (5) SEQ ID NO: 27, (6) SEQ ID NO: 28, (7) both of SEQ ID NOs: 19 and 20, (8) both of SEQ ID NOs: 25 and 26, or (9) both of SEQ ID NOs: 27 and 28, wherein all of SEQ ID NOs: 19-20 and 25-28 are as defined in Table 4.

TABLE 4

| SEQ ID NO: | Definitions of “X” |
|---|---|
| SEQ ID NO: 19 | X at position 48 is Cys; |
| (constant region α | X at position 112 is Ser, Ala, Val, Leu, Ile, Pro, Phe, Met, or Trp; |
| chain) | preferably wherein X at position 112 is Leu, Ile, or Val; |
| | especially preferably wherein X at position 112 is Leu; |
| | X at position 114 is Met, Ala, Val, Leu, Ile, Pro, Phe, or Trp; |
| | preferably wherein X at position 114 is Leu, Ile, or Val; |
| | especially preferably wherein X at position 114 is Ile; and |
| | X at position 115 is Gly, Ala, Val, Leu, Ile, Pro, Phe, Met, or Trp; |
| | preferably wherein X at position 115 is Leu, Ile, or Val; and |
| | especially preferably wherein X at position 115 is Val, |
| | wherein SEQ ID NO: 19 does not simultaneously comprise all of Ser at |
| | position 112, Met at position 114, and Gly at position 115. |
| SEQ ID NO: 20 | X at position 57 is Cys |
| (constant region β | |
| chain) | |
| SEQ ID NO: 25 (α | X at position 185 is Cys; |
| chain) (with N-terminal | X at position 249 is Ser, Ala, Val, Leu, Ile, Pro, Phe, Met, or Trp; |
| signal peptide) | preferably wherein X at position 249 is Leu, Ile, or Val; |
| | especially preferably wherein X at position 249 is Leu; |
| | X at position 251 is Met, Ala, Val, Leu, Ile, Pro, Phe, or Trp; |
| | preferably wherein X at position 251 is Leu, Ile, or Val; |
| | especially preferably wherein X at position 251 is Ile; and |
| | X at position 252 is Gly, Ala, Val, Leu, Ile, Pro, Phe, Met, or Trp; |
| | preferably wherein X at position 252 is Leu, Ile, or Val; and |
| | especially preferably wherein X at position 252 is Val, |
| | wherein SEQ ID NO: 25 does not simultaneously comprise all of Ser at |
| | position 249, Met at position 251, and Gly at position 252. |
| SEQ ID NO: 26 (β | X at position 186 is Cys |
| chain) (with N-terminal | |
| signal peptide) | |
| SEQ ID NO: 27 (α | X at position 165 is Cys; X at position |
| chain) (predicted | 229 is Ser, Ala, Val, Leu, Ile, Pro, Phe, Met, or Trp; |
| sequence without N- | preferably wherein X at position 229 is Leu, Ile, or Val; |
| terminal signal peptide) | especially preferably wherein X at position 229 is Leu; |
| | X at position 231 is Met, Ala, Val, Leu, Ile, Pro, Phe, or Trp; |
| | preferably wherein X at position 231 is Leu, Ile, or Val; |
| | especially preferably wherein X at position 231 is Ile; and |
| | X at position 232 is Gly, Ala, Val, Leu, Ile, Pro, Phe, Met, or Trp; |
| | preferably wherein X at position 232 is Leu, Ile, or Val; and |
| | especially preferably wherein X at position 232 is Val, |
| | wherein SEQ ID NO: 27 does not simultaneously comprise all of Ser at |
| | position 229, Met at position 231, and Gly at position 232. |
| SEQ ID NO: 28 (β | X at position 167 is Cys |
| chain) (predicted | |
| sequence without N- | |
| terminal signal peptide) | |

In an aspect of the invention, the cysteine-substituted, LVL-modified TCR comprises (a) SEQ ID NO: 21 (α chain constant region of cysteine-substituted, LVL-modified TCR); (b) SEQ ID NO: 22 (β chain constant region of cysteine-substituted, LVL-modified TCR); or (c) both (a) and (b).

Also provided by the invention is a polypeptide comprising a functional portion of any of the TCRs described herein. The term “polypeptide,” as used herein, includes oligopeptides and refers to a single chain of amino acids connected by one or more peptide bonds.

With respect to the inventive polypeptides, the functional portion can be any portion comprising contiguous amino acids of the TCR of which it is a part, provided that the functional portion specifically binds to Q61K RAS. The term “functional portion,” when used in reference to a TCR, refers to any part or fragment of the TCR of the invention, which part or fragment retains the biological activity of the TCR of which it is a part (the parent TCR). Functional portions encompass, for example, those parts of a TCR that retain the ability to specifically bind to Q61K RAS (e.g., within the context of any of the HLA Class I molecules described herein), or detect, treat, or prevent cancer, to a similar extent, the same extent, or to a higher extent, as the parent TCR. In reference to the parent TCR, the functional portion can comprise, for instance, about 10%, about 25%, about 30%, about 50%, about 70%, about 80%, about 90%, about 95%, or more, of the parent TCR.

The functional portion can comprise additional amino acids at the amino or carboxy terminus of the portion, or at both termini, which additional amino acids are not found in the amino acid sequence of the parent TCR. Desirably, the additional amino acids do not interfere with the biological function of the functional portion, e.g., specifically binding to Q61K RAS; and/or having the ability to detect cancer, treat or prevent cancer, etc. More desirably, the additional amino acids enhance the biological activity, as compared to the biological activity of the parent TCR.

The polypeptide can comprise a functional portion of either or both of the α and β chains of the TCRs of the invention, such as a functional portion comprising one or more of the CDR1, CDR2, and CDR3 of the variable region(s) of the α chain and/or β chain of a TCR of the invention. In an aspect of the invention, the polypeptide can comprise the amino acid sequence of SEQ ID NO: 1 (CDR1 of α chain), SEQ ID NO: 2 (CDR2 of α chain), SEQ ID NO: 3 (CDR3 of α chain), SEQ ID NO: 4 (CDR1 of β chain), SEQ ID NO: 5 (CDR2 of β chain), or SEQ ID NO: 6 (CDR3 of β chain). In this regard, the inventive polypeptide can comprise any one or more of the amino acid sequences selected from the group consisting of SEQ ID NOs: 1-6. In an aspect of the invention, the polypeptide comprises the amino acid sequences of: (a) all of SEQ ID NOs: 1-3, (b) all of SEQ ID NOs: 4-6, or (c) all of SEQ ID NOs: 1-6. In a preferred aspect, the polypeptide comprises the amino acid sequences of all of SEQ ID NOs: 1-6.

In an aspect of the invention, the inventive polypeptide can comprise, for instance, the variable region of the inventive TCR comprising a combination of the CDR regions set forth above. In this regard, the polypeptide can comprise the amino acid sequence(s) of (1) SEQ ID NO: 7, (2) SEQ ID NO: 8, (3) SEQ ID NO: 9, (4) SEQ ID NO: 10, (5) both of SEQ ID NOs: 7 and 8, or (6) both of SEQ ID NOs: 9 and 10. In a preferred aspect, the polypeptide comprises the amino acid sequences of (a) both of SEQ ID NOs: 7 and 8 or (b) both of SEQ ID NOs: 9 and 10.

In an aspect of the invention, the inventive polypeptide can further comprise the constant region of the inventive TCR set forth above. In this regard, the polypeptide can further comprise the amino acid sequence of SEQ ID NO: 23 (WT murine constant region of α chain), SEQ ID NO: 24 (WT murine constant region of β chain), SEQ ID NO: 19 (substituted murine constant region of α chain), SEQ ID NO: 20 (substituted murine constant region of β chain), SEQ ID NO: 21 (α chain constant region of cysteine-substituted, LVL-modified TCR); SEQ ID NO: 22 (β chain constant region of cysteine-substituted, LVL-modified TCR); both SEQ ID NOs: 19 and 20, both SEQ ID NOs: 21 and 22, or both SEQ ID NOs: 23 and 24. Preferably, the polypeptide further comprises the amino acid sequences of both of SEQ ID NOs: 19 and 20, both of SEQ ID NO: 21 and 22, or both of SEQ ID NOs: 23 and 24 in combination with any of the CDR regions or variable regions described herein with respect to other aspects of the invention. In an aspect of the invention, one or both of SEQ ID NOs: 19 and 20 of the polypeptide are as defined in any one of Tables 2-4.

In an aspect of the invention, the inventive polypeptide can comprise the entire length of an α or β chain of the TCR described herein. In this regard, the inventive polypeptide can comprise the amino acid sequence of SEQ ID NO: 25, SEQ ID NO: 26, SEQ ID NO: 27, SEQ ID NO: 28, SEQ ID NO: 29, SEQ ID NO: 30, SEQ ID NO: 31, or SEQ ID NO: 32. Alternatively, the polypeptide of the invention can comprise both chains of the TCRs described herein. For example, the polypeptide may comprise the amino acid sequences of: both of SEQ ID NOs: 25-26, both of SEQ ID NOs: 27-28, both of SEQ ID NOs: 29-30, or both of SEQ ID NOs: 31-32.

For example, the polypeptide of the invention can comprise (a) the amino acid sequence of SEQ ID NO: 25 (α chain with N-terminal signal peptide), wherein: (i) X at position 185 of SEQ ID NO: 25 is Thr or Cys; (ii) X at position 249 of SEQ ID NO: 25 is Ser, Ala, Val, Leu, Ile, Pro, Phe, Met, or Trp; (iii) X at position 251 of SEQ ID NO: 25 is Met, Ala, Val, Leu, Ile, Pro, Phe, or Trp; and (iv) X at position 252 of SEQ ID NO: 25 is Gly, Ala, Val, Leu, Ile, Pro, Phe, Met, or Trp; (b) the amino acid sequence of SEQ ID NO: 26 (β chain with N-terminal signal peptide), wherein X at position 186 of SEQ ID NO: 26 is Ser or Cys; (c) the amino acid sequences of both of SEQ ID NOs: 25 and 26; (d) the amino acid sequence of SEQ ID NO: 27 (predicted sequence of α chain without N-terminal signal peptide), wherein: (i) X at position 165 of SEQ ID NO: 27 is Thr or Cys; (ii) X at position 229 of SEQ ID NO: 27 is Ser, Ala, Val, Leu, Ile, Pro, Phe, Met, or Trp; (iii) X at position 231 of SEQ ID NO: 27 is Met, Ala, Val, Leu, Ile, Pro, Phe, or Trp; and (iv) X at position 232 of SEQ ID NO: 27 is Gly, Ala, Val, Leu, Ile, Pro, Phe, Met, or Trp; (e) the amino acid sequence of SEQ ID NO: 28 (predicted sequence of β chain without N-terminal signal peptide), wherein X at position 167 of SEQ ID NO: 28 is Ser or Cys; (f) the amino acid sequences of both of SEQ ID NOs: 27 and 28; (g) the amino acid sequence of SEQ ID NO: 29 (α chain of cysteine-substituted, LVL-modified TCR with N-terminal signal peptide); (h) the amino acid sequence of SEQ ID NO: 30 (β chain of cysteine-substituted, LVL-modified TCR with N-terminal signal peptide); (i) the amino acid sequence of SEQ ID NO: 31 (predicted sequence of α chain of cysteine-substituted, LVL-modified TCR without N-terminal signal peptide); (j) the amino acid sequence of SEQ ID NO: 32 (predicted sequence of β chain of cysteine-substituted, LVL-modified TCR without N-terminal signal peptide); (k) the amino acid sequences of both of SEQ ID NOs: 29 and 30; or (l) the amino acid sequences of both of SEQ ID NOs: 31 and 32. In an aspect of the invention, one or more of SEQ ID NOs: 25-28 of the polypeptide are as defined in any one of Tables 2-4.

An aspect of the invention provides a protein comprising at least one of the polypeptides described herein. By “protein” is meant a molecule comprising one or more polypeptide chains.

In an aspect of the invention, the protein of the invention can comprise first and second polypeptide chains, wherein: (a) the first polypeptide chain comprises the amino acid sequences of SEQ ID NOs: 1-3; (b) the second polypeptide chain comprises the amino acid sequences of SEQ ID NOs: 4-6; and (c) both (a) and (b).

In another aspect of the invention, (i) the first polypeptide chain comprises the amino acid sequence of SEQ ID NO: 7; (ii) the second polypeptide chain comprises the amino acid sequence of SEQ ID NO: 8; (iii) the first polypeptide chain comprises the amino acid sequence of SEQ ID NO: 9; (iv) the second polypeptide chain comprises the amino acid sequence of SEQ ID NO: 10; (v) both (i) and (ii); or (vi) both (iii) and (iv).

The inventive protein may further comprise any of the constant regions described herein with respect to other aspects of the invention. In this regard, in an aspect of the invention, (i) the first polypeptide chain may further comprise the amino acid sequence of SEQ ID NO: 19 and the second polypeptide chain may further comprise the amino acid sequence of SEQ ID NO: 20; (ii) the first polypeptide chain may further comprise the amino acid sequence of SEQ ID NO: 21 and the second polypeptide chain may further comprise the amino acid sequence of SEQ ID NO: 22; or (ii) the first polypeptide chain may comprise the amino acid sequence of SEQ ID NO: 23 and the second polypeptide chain may comprise the amino acid sequence of SEQ ID NO: 24. In an aspect of the invention, one or both of SEQ ID NOs: 19 and 20 of the protein are as defined in any one of Tables 2-4.

The inventive protein may comprise a full length α or β chain, as described herein with respect to other aspects of the invention. In this regard, in an aspect of the invention, (a) the first polypeptide chain comprises the amino acid sequence of SEQ ID NO: 25, wherein: (i) X at position 185 of SEQ ID NO: 25 is Thr or Cys; (ii) X at position 249 of SEQ ID NO: 25 is Ser, Ala, Val, Leu, Ile, Pro, Phe, Met, or Trp; (iii) X at position 251 of SEQ ID NO: 25 is Met, Ala, Val, Leu, Ile, Pro, Phe, or Trp; and (iv) X at position 252 of SEQ ID NO: 25 is Gly, Ala, Val, Leu, Ile, Pro, Phe, Met, or Trp; (b) the second polypeptide chain comprises the amino acid sequence of SEQ ID NO: 26, wherein X at position 186 of SEQ ID NO: 26 is Ser or Cys; (c) both (a) and (b); (d) the first polypeptide chain comprises the amino acid sequence of SEQ ID NO: 27, wherein (i) X at position 165 of SEQ ID NO: 27 is Thr or Cys; (ii) X at position 229 of SEQ ID NO: 27 is Ser, Ala, Val, Leu, Ile, Pro, Phe, Met, or Trp; (iii) X at position 231 of SEQ ID NO: 27 is Met, Ala, Val, Leu, Ile, Pro, Phe, or Trp; and (iv) X at position 232 of SEQ ID NO: 27 is Gly, Ala, Val, Leu, Ile, Pro, Phe, Met, or Trp; (e) the second polypeptide chain comprises the amino acid sequence of SEQ ID NO: 28, wherein X at position 167 of SEQ ID NO: 28 is Ser or Cys; (f) both (d) and (e); (g) the first polypeptide chain comprises the amino acid sequence of SEQ ID NO: 29; (h) the second polypeptide chain comprises the amino acid sequence of SEQ ID NO: 30; (i) the first polypeptide chain comprises the amino acid sequence of SEQ ID NO: 31; (j) the second polypeptide chain comprises the amino acid sequence of SEQ ID NO: 32; (k) both (g) and (h); or (l) both (i) and (j). In an aspect of the invention, one or more of SEQ ID NOs: 25-28 of the protein are as defined in any one of Tables 2-4.

The protein of the invention can be a TCR. Alternatively, if, for example, the protein comprises a single polypeptide chain comprising the amino acid sequences of both the TCR α and β chains, or if the first and/or second polypeptide chain(s) of the protein further comprise(s) other amino acid sequences, e.g., an amino acid sequence encoding an immunoglobulin or a portion thereof, then the inventive protein can be a fusion protein. In this regard, the invention also provides a fusion protein comprising at least one of the inventive polypeptides described herein along with at least one other polypeptide. The other polypeptide can exist as a separate polypeptide of the fusion protein, or can exist as a polypeptide, which is expressed in frame (in tandem) with one of the inventive polypeptides described herein. The other polypeptide can encode any peptidic or proteinaceous molecule, or a portion thereof, including, but not limited to an immunoglobulin, CD3, CD4, CD8, an MHC molecule, a CD1 molecule, e.g., CD1a, CD1b, CD1c, CD1d, etc.

The fusion protein can comprise one or more copies of the inventive polypeptide and/or one or more copies of the other polypeptide. For instance, the fusion protein can comprise 1, 2, 3, 4, 5, or more, copies of the inventive polypeptide and/or of the other polypeptide. Suitable methods of making fusion proteins are known in the art, and include, for example, recombinant methods.

In some aspects of the invention, the TCRs, polypeptides, and proteins of the invention may be expressed as a single protein comprising a linker peptide linking the α chain and the β chain. In this regard, the TCRs, polypeptides, and proteins of the invention may further comprise a linker peptide. The linker peptide may advantageously facilitate the expression of a recombinant TCR, polypeptide, and/or protein in a host cell. The linker peptide may comprise any suitable amino acid sequence. The linker peptide may be a cleavable linker peptide. For example, the linker peptide may be a furin-SGSG-P2A linker peptide comprising the amino acid sequence of RAKRSGSGATNFSLLKQAGDVEENPGP (SEQ ID NO: 45). Upon expression of the construct including the linker peptide by a host cell, the linker peptide may be cleaved, resulting in separated α and β chains. In an aspect of the invention, the TCR, polypeptide, or protein may comprise an amino acid sequence comprising a full-length α chain, a full-length β chain, and a linker peptide positioned between the α and β chains.

The protein of the invention can be a recombinant antibody, or an antigen binding portion thereof, comprising at least one of the inventive polypeptides described herein. As used herein, “recombinant antibody” refers to a recombinant (e.g., genetically engineered) protein comprising at least one of the polypeptides of the invention and a polypeptide chain of an antibody, or an antigen binding portion thereof. The polypeptide of an antibody, or antigen binding portion thereof, can be a heavy chain, a light chain, a variable or constant region of a heavy or light chain, a single chain variable fragment (scFv), or an Fc, Fab, or F(ab)₂′ fragment of an antibody, etc. The polypeptide chain of an antibody, or an antigen binding portion thereof, can exist as a separate polypeptide of the recombinant antibody. Alternatively, the polypeptide chain of an antibody, or an antigen binding portion thereof, can exist as a polypeptide, which is expressed in frame (in tandem) with the polypeptide of the invention. The polypeptide of an antibody, or an antigen binding portion thereof, can be a polypeptide of any antibody or any antibody fragment, including any of the antibodies and antibody fragments described herein.

Included in the scope of the invention are functional variants of the inventive TCRs, polypeptides, or proteins described herein. The term “functional variant,” as used herein, refers to a TCR, polypeptide, or protein having substantial or significant sequence identity or similarity to a parent TCR, polypeptide, or protein, which functional variant retains the biological activity of the TCR, polypeptide, or protein of which it is a variant. Functional variants encompass, for example, those variants of the TCR, polypeptide, or protein described herein (the parent TCR, polypeptide, or protein) that retain the ability to specifically bind to the Q61K RAS for which the parent TCR has antigenic specificity or to which the parent polypeptide or protein specifically binds, to a similar extent, the same extent, or to a higher extent, as the parent TCR, polypeptide, or protein. In reference to the parent TCR, polypeptide, or protein, the functional variant can, for instance, be at least about 30%, about 50%, about 75%, about 80%, about 90%, about 95%, about 96%, about 97%, about 98%, about 99% or more identical in amino acid sequence to the parent TCR, polypeptide, or protein, respectively.

The functional variant can, for example, comprise the amino acid sequence of the parent TCR, polypeptide, or protein with at least one conservative amino acid substitution. Conservative amino acid substitutions are known in the art, and include amino acid substitutions in which one amino acid having certain physical and/or chemical properties is exchanged for another amino acid that has the same chemical or physical properties. For instance, the conservative amino acid substitution can be an acidic amino acid substituted for another acidic amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Val, etc.), a basic amino acid substituted for another basic amino acid (Lys, Arg, etc.), an amino acid with a polar side chain substituted for another amino acid with a polar side chain (Asn, Cys, Gln, Ser, Thr, Tyr, etc.), etc.

Alternatively or additionally, the functional variants can comprise the amino acid sequence of the parent TCR, polypeptide, or protein with at least one non-conservative amino acid substitution. In this case, it is preferable for the non-conservative amino acid substitution to not interfere with or inhibit the biological activity of the functional variant. Preferably, the non-conservative amino acid substitution enhances the biological activity of the functional variant, such that the biological activity of the functional variant is increased as compared to the parent TCR, polypeptide, or protein.

The TCR, polypeptide, or protein can consist essentially of the specified amino acid sequence or sequences described herein, such that other components of the TCR, polypeptide, or protein, e.g., other amino acids, do not materially change the biological activity of the TCR, polypeptide, or protein. In this regard, the inventive TCR, polypeptide, or protein can, for example, consist essentially of the amino acid sequence of SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, both of SEQ ID NOs: 25-26, both of SEQ ID NOs: 27-28, both of SEQ ID NOs: 29-30, or both of SEQ ID NOs: 31-32. Also, for instance, the inventive TCRs, polypeptides, or proteins can consist essentially of the amino acid sequence(s) of SEQ ID NO: 7, SEQ ID NO: 8, SEQ ID NO: 9, SEQ ID NO: 10, both of SEQ ID NOs: 7 and 8, or both of SEQ ID NOs: 9 and 10. Furthermore, the inventive TCRs, polypeptides, or proteins can consist essentially of the amino acid sequences of (a) all of SEQ ID NOs: 1-3, (b) all of SEQ ID NOs: 4-6, or (c) all of SEQ ID NOs: 1-6. The TCRs, polypeptides, and proteins of the invention can be of any length, i.e., can comprise any number of amino acids, provided that the TCRs, polypeptides, or proteins retain their biological activity, e.g., the ability to specifically bind to Q61K RAS; detect cancer in a mammal; or treat or prevent cancer in a mammal, etc. For example, the polypeptide can be in the range of from about 50 to about 5000 amino acids long, such as about 50, about 70, about 75, about 100, about 125, about 150, about 175, about 200, about 300, about 400, about 500, about 600, about 700, about 800, about 900, about 1000 or more amino acids in length. In this regard, the polypeptides of the invention also include oligopeptides.

The TCRs, polypeptides, and proteins of the invention can comprise synthetic amino acids in place of one or more naturally-occurring amino acids. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α,β-diaminopropionic acid, homophenylalanine, and α-tert-butylglycine.

The TCRs, polypeptides, and proteins of the invention can be glycosylated, amidated, carboxylated, phosphorylated, esterified, N-acylated, cyclized via, e.g., a disulfide bridge, or converted into an acid addition salt and/or optionally dimerized or polymerized, or conjugated.

The TCR, polypeptide, and/or protein of the invention can be obtained by methods known in the art such as, for example, de novo synthesis. Also, polypeptides and proteins can be recombinantly produced using the nucleic acids described herein using standard recombinant methods. See, for instance, Green and Sambrook, Molecular Cloning: A Laboratory Manual, 4^{th} ed., Cold Spring Harbor Press, Cold Spring Harbor, NY (2012). Alternatively, the TCRs, polypeptides, and/or proteins described herein can be synthesized by any of a variety of commercial entities. In this respect, the inventive TCRs, polypeptides, and proteins can be synthetic, recombinant, isolated, and/or purified. An aspect of the invention provides an isolated or purified TCR, polypeptide, or protein encoded by any of the inventive nucleic acids or vectors described herein with respect to other aspects of the invention. Another aspect of the invention provides an isolated or purified TCR, polypeptide, or protein that results from expression of any of the inventive nucleic acids or vectors described herein in a cell. Still another aspect of the invention provides a method of producing any of the inventive TCRs, polypeptides, or proteins described herein, the method comprising culturing any of the inventive host cells or populations of host cells described herein so that the TCR, polypeptide, or protein is produced.

Included in the scope of the invention are conjugates, e.g., bioconjugates, comprising any of the inventive TCRs, polypeptides, or proteins (including any of the functional portions or variants thereof), nucleic acids, recombinant expression vectors, host cells, populations of host cells, or antibodies, or antigen binding portions thereof. Conjugates, as well as methods of synthesizing conjugates in general, are known in the art.

An aspect of the invention provides a nucleic acid comprising a nucleotide sequence encoding any of the TCRs, polypeptides, or proteins described herein. “Nucleic acid.” as used herein, includes “polynucleotide,” “oligonucleotide,” and “nucleic acid molecule.” and generally means a polymer of DNA or RNA, which can be single-stranded or double-stranded, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. In an aspect, the nucleic acid comprises complementary DNA (cDNA). It is generally preferred that the nucleic acid does not comprise any insertions, deletions, inversions, and/or substitutions. However, it may be suitable in some instances, as discussed herein, for the nucleic acid to comprise one or more insertions, deletions, inversions, and/or substitutions.

Preferably, the nucleic acids of the invention are recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication.

The nucleic acids can be constructed based on chemical synthesis and/or enzymatic ligation reactions using procedures known in the art. See, for example, Green and Sambrook et al., supra. For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, β-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N6-substituted adenine, 7-methylguanine, 5-methylaminomethyluracil, 5-methoxyaminomethyl-2-thiouracil, β-D-mannosylqueosine, 5′-methoxycarboxymethyluracil, 5-methoxyuracil, 2-methylthio-N⁶-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the invention can be purchased from any of a variety of commercial entities.

The nucleic acid can comprise any nucleotide sequence which encodes any of the TCRs, polypeptides, or proteins described herein. In an aspect of the invention, the nucleic acid comprises a codon-optimized nucleotide sequence encoding any of the TCRs, polypeptides, or proteins described herein. Without being bound to any particular theory or mechanism, it is believed that codon optimization of the nucleotide sequence increases the translation efficiency of the mRNA transcripts. Codon optimization of the nucleotide sequence may involve substituting a native codon for another codon that encodes the same amino acid, but can be translated by tRNA that is more readily available within a cell, thus increasing translation efficiency. Optimization of the nucleotide sequence may also reduce secondary mRNA structures that would interfere with translation, thus increasing translation efficiency. In an aspect of the invention, the nucleic acid comprises the nucleotide sequence of SEQ ID NO: 42 (codon-optimized nucleotide sequence encoding α chain variable region with N-terminal signal peptide), SEQ ID NO: 43 (codon-optimized nucleotide sequence encoding β chain variable region with N-terminal signal peptide), or both SEQ ID NOs: 42 and 43.

The invention also provides a nucleic acid comprising a nucleotide sequence which is complementary to the nucleotide sequence of any of the nucleic acids described herein or a nucleotide sequence which hybridizes under stringent conditions to the nucleotide sequence of any of the nucleic acids described herein.

The nucleotide sequence which hybridizes under stringent conditions preferably hybridizes under high stringency conditions. By “high stringency conditions” is meant that the nucleotide sequence specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-10 bases) that matched the nucleotide sequence. Such small regions of complementarity are more easily melted than a full-length complement of 14-17 or more bases, and high stringency hybridization makes them easily distinguishable. Relatively high stringency conditions would include, for example, low salt and/or high temperature conditions, such as provided by about 0.02-0.1 M NaCl or the equivalent, at temperatures of about 50-70° C. Such high stringency conditions tolerate little, if any, mismatch between the nucleotide sequence and the template or target strand, and are particularly suitable for detecting expression of any of the inventive TCRs. It is generally appreciated that conditions can be rendered more stringent by the addition of increasing amounts of formamide.

An aspect of the invention also provides a nucleic acid comprising a nucleotide sequence that is at least about 70% or more, e.g., about 80%, about 90%, about 91%, about 92%, about 93%, about 94%, about 95%, about 96%, about 97%, about 98%, or about 99% identical to any of the nucleic acids described herein. In this regard, the nucleic acid may consist essentially of any of the nucleotide sequences described herein.

An aspect of the invention provides an isolated or purified nucleic acid comprising, from 5′ to 3′, a first nucleic acid sequence and a second nucleotide sequence, wherein the first and second nucleotide sequence, respectively, encode the amino sequences of SEQ ID NOs: 7 and 8; 8 and 7; 9 and 10; 10 and 9; 25 and 26; 26 and 25; 27 and 28; 28 and 27; 29 and 30; 30 and 29; 31 and 32; or 32 and 31.

In an aspect of the invention, the isolated or purified nucleic acid further comprises a third nucleotide sequence interposed between the first and second nucleotide sequence, wherein the third nucleotide sequence encodes a cleavable linker peptide. In an aspect of the invention, the cleavable linker peptide comprises the amino acid sequence of

(SEQ ID NO: 45)
RAKRSGSGATNFSLLKQAGDVEENPGP.

The nucleic acids of the invention can be incorporated into a recombinant expression vector. In this regard, the invention provides a recombinant expression vector comprising any of the nucleic acids of the invention. In an aspect of the invention, the recombinant expression vector comprises a nucleotide sequence encoding the α chain, the β chain, and linker peptide.

For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors of the invention are not naturally-occurring as a whole. However, parts of the vectors can be naturally-occurring. The inventive recombinant expression vectors can comprise any type of nucleotide, including, but not limited to DNA and RNA, which can be single-stranded or double-stranded, synthesized or obtained in part from natural sources, and which can contain natural, non-natural or altered nucleotides. The recombinant expression vectors can comprise naturally-occurring, non-naturally-occurring internucleotide linkages, or both types of linkages. Preferably, the non-naturally occurring or altered nucleotides or internucleotide linkages do not hinder the transcription or replication of the vector.

The recombinant expression vector of the invention can be any suitable recombinant expression vector, and can be used to transform or transfect any suitable host cell. Suitable vectors include those designed for propagation and expansion or for expression or both, such as plasmids and viruses. The vector can be selected from the group consisting of the pUC series (Fermentas Life Sciences), the pBluescript series (Stratagene, LaJolla, CA), the pET series (Novagen, Madison, WI), the pGEX series (Pharmacia Biotech, Uppsala, Sweden), and the pEX series (Clontech, Palo Alto, CA). Bacteriophage vectors, such as λGT10, λGT11, λZapII (Stratagene), λEMBL4, and λNM1149, also can be used. Examples of plant expression vectors include pBI01, pBI101.2, pBI101.3, pBI121 and pBIN19 (Clontech). Examples of animal expression vectors include pEUK-C1, pMAM and pMAMneo (Clontech). Preferably, the recombinant expression vector is a viral vector, e.g., a retroviral vector. In an especially preferred aspect, the recombinant expression vector is an MSGV1 vector. In an aspect of the invention, the recombinant expression vector is a transposon or a lentiviral vector.

The recombinant expression vectors of the invention can be prepared using standard recombinant DNA techniques described in, for example, Green and Sambrook et al., supra. Constructs of expression vectors, which are circular or linear, can be prepared to contain a replication system functional in a prokaryotic or eukaryotic host cell. Replication systems can be derived, e.g., from ColE1, 2μ plasmid, λ, SV40, bovine papillomavirus, and the like.

Desirably, the recombinant expression vector comprises regulatory sequences, such as transcription and translation initiation and termination codons, which are specific to the type of host cell (e.g., bacterium, fungus, plant, or animal) into which the vector is to be introduced, as appropriate and taking into consideration whether the vector is DNA- or RNA-based.

The recombinant expression vector can include one or more marker genes, which allow for selection of transformed or transfected host cells. Marker genes include biocide resistance, e.g., resistance to antibiotics, heavy metals, etc., complementation in an auxotrophic host cell to provide prototrophy, and the like. Suitable marker genes for the inventive expression vectors include, for instance, neomycin/G418 resistance genes, hygromycin resistance genes, histidinol resistance genes, tetracycline resistance genes, and ampicillin resistance genes.

The recombinant expression vector can comprise a native or nonnative promoter operably linked to the nucleotide sequence encoding the TCR, polypeptide, or protein, or to the nucleotide sequence which is complementary to or which hybridizes to the nucleotide sequence encoding the TCR, polypeptide, or protein. The selection of promoters, e.g., strong, weak, inducible, tissue-specific and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an SV40 promoter, an RSV promoter, and a promoter found in the long-terminal repeat of the murine stem cell virus.

The inventive recombinant expression vectors can be designed for either transient expression, for stable expression, or for both. Also, the recombinant expression vectors can be made for constitutive expression or for inducible expression.

Further, the recombinant expression vectors can be made to include a suicide gene. As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art and include, for example, the Herpes Simplex Virus (HSV) thymidine kinase (TK) gene, cytosine deaminase, purine nucleoside phosphorylase, nitroreductase, and the inducible caspase 9 gene system.

Another aspect of the invention further provides a host cell comprising any of the nucleic acids or recombinant expression vectors described herein. As used herein, the term “host cell” refers to any type of cell that can contain the inventive recombinant expression vector. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DH5α E. coli cells, Chinese hamster ovarian cells, monkey VERO cells, COS cells, HEK293 cells, and the like. For purposes of amplifying or replicating the recombinant expression vector, the host cell is preferably a prokaryotic cell, e.g., a DH5α cell. For purposes of producing a recombinant TCR, polypeptide, or protein, the host cell is preferably a mammalian cell. Most preferably, the host cell is a human cell. While the host cell can be of any cell type, can originate from any type of tissue, and can be of any developmental stage, the host cell preferably is a peripheral blood lymphocyte (PBL) or a peripheral blood mononuclear cell (PBMC). More preferably, the host cell is a T cell. In an aspect of the invention, the host cell is a human lymphocyte. In another aspect of the invention, the host cell is selected from the group consisting of a T cell, a natural killer T (NKT) cell, an invariant natural killer T (INKT) cell, and a natural killer (NK) cell. Still another aspect of the invention provides a method of producing a host cell expressing a TCR that has antigenic specificity for the peptide of ILDTAGKEEY (SEQ ID NO: 37), the method comprising contacting a cell with any of the recombinant expression vectors described herein under conditions that allow introduction of the recombinant expression vector into the cell.

For purposes herein, the T cell can be any T cell, such as a cultured T cell, e.g., a primary T cell, or a T cell from a cultured T cell line, e.g., Jurkat, SupT1, etc., or a T cell obtained from a mammal. If obtained from a mammal, the T cell can be obtained from numerous sources, including but not limited to blood, bone marrow, lymph node, the thymus, or other tissues or fluids. T cells can also be enriched for or purified. Preferably, the T cell is a human T cell. The T cell can be any type of T cell and can be of any developmental stage, including but not limited to, CD4⁺/CD8⁺ double positive T cells, CD4⁺ helper T cells, e.g., Th₁ and Th₂ cells, CD4⁺ T cells, CD8⁺ T cells (e.g., cytotoxic T cells), tumor infiltrating lymphocytes (TILs), memory T cells (e.g., central memory T cells and effector memory T cells), naïve T cells, and the like.

Also provided by the invention is a population of cells comprising at least one host cell described herein. The population of cells can be a heterogeneous population comprising the host cell comprising any of the recombinant expression vectors described, in addition to at least one other cell, e.g., a host cell (e.g., a T cell), which does not comprise any of the recombinant expression vectors, or a cell other than a T cell, e.g., a B cell, a macrophage, a neutrophil, an erythrocyte, a hepatocyte, an endothelial cell, an epithelial cells, a muscle cell, a brain cell, etc. Alternatively, the population of cells can be a substantially homogeneous population, in which the population comprises mainly of host cells (e.g., consisting essentially of) comprising the recombinant expression vector. The population also can be a clonal population of cells, in which all cells of the population are clones of a single host cell comprising a recombinant expression vector, such that all cells of the population comprise the recombinant expression vector. In one aspect of the invention, the population of cells is a clonal population comprising host cells comprising a recombinant expression vector as described herein.

In an aspect of the invention, the numbers of cells in the population may be rapidly expanded. Expansion of the numbers of T cells can be accomplished by any of a number of methods as are known in the art as described in, for example, U.S. Pat. Nos. 8,034,334; 8,383,099; U.S. Patent Application Publication No. 2012/0244133; Dudley et al., J. Immunother., 26:332-42 (2003); and Riddell et al., J. Immunol. Methods, 128:189-201 (1990). In an aspect, expansion of the numbers of T cells is carried out by culturing the T cells with OKT3 antibody, IL-2, and feeder PBMC (e.g., irradiated allogeneic PBMC).

The inventive TCRs, polypeptides, proteins, nucleic acids, recombinant expression vectors, and host cells (including populations thereof), can be isolated and/or purified. The term “isolated,” as used herein, means having been removed from its natural environment. The term “purified,” as used herein, means having been increased in purity, wherein “purity” is a relative term, and not to be necessarily construed as absolute purity. For example, the purity can be at least about 50%, can be greater than about 60%, about 70%, about 80%, about 90%, about 95%, or can be about 100%.

The inventive TCRs, polypeptides, proteins, nucleic acids, recombinant expression vectors, and host cells (including populations thereof), all of which are collectively referred to as “inventive TCR materials” hereinafter, can be formulated into a composition, such as a pharmaceutical composition. In this regard, the invention provides a pharmaceutical composition comprising any of the TCRs, polypeptides, proteins, nucleic acids, recombinant expression vectors, and host cells (including populations thereof), described herein, and a pharmaceutically acceptable carrier. The inventive pharmaceutical compositions containing any of the inventive TCR materials can comprise more than one inventive TCR material, e.g., a polypeptide and a nucleic acid, or two or more different TCRs. Alternatively, the pharmaceutical composition can comprise an inventive TCR material in combination with another pharmaceutically active agent(s) or drug(s), such as a chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc.

Preferably, the carrier is a pharmaceutically acceptable carrier. With respect to pharmaceutical compositions, the carrier can be any of those conventionally used for the particular inventive TCR material under consideration. Methods for preparing administrable compositions are known or apparent to those skilled in the art and are described in more detail in, for example, Remington: The Science and Practice of Pharmacy, 22^{nd} Ed., Pharmaceutical Press (2012). It is preferred that the pharmaceutically acceptable carrier be one which has no detrimental side effects or toxicity under the conditions of use.

The choice of carrier will be determined in part by the particular inventive TCR material, as well as by the particular method used to administer the inventive TCR material. Accordingly, there are a variety of suitable formulations of the pharmaceutical composition of the invention. Suitable formulations may include any of those for parenteral, subcutaneous, intravenous, intramuscular, intraarterial, intrathecal, intratumoral, or interperitoneal administration. More than one route can be used to administer the inventive TCR materials, and in certain instances, a particular route can provide a more immediate and more effective response than another route.

Preferably, the inventive TCR material is administered by injection, e.g., intravenously. When the inventive TCR material is a host cell (or population thereof) expressing the inventive TCR, the pharmaceutically acceptable carrier for the cells for injection may include any isotonic carrier such as, for example, normal saline (about 0.90% w/v of NaCl in water, about 300 mOsm/L NaCl in water, or about 9.0 g NaCl per liter of water), NORMOSOL R electrolyte solution (Abbott, Chicago, IL), PLASMA-LYTE A (Baxter, Deerfield, IL), about 5% dextrose in water, or Ringer's lactate. In an aspect, the pharmaceutically acceptable carrier is supplemented with human serum albumen.

For purposes of the invention, the amount or dose (e.g., numbers of cells when the inventive TCR material is one or more cells) of the inventive TCR material administered should be sufficient to effect, e.g., a therapeutic or prophylactic response, in the subject or animal over a reasonable time frame. For example, the dose of the inventive TCR material should be sufficient to bind to a cancer antigen (e.g., Q61K RAS), or detect, treat or prevent cancer in a period of from about 2 hours or longer, e.g., 12 to 24 or more hours, from the time of administration. In certain aspects, the time period could be even longer. The dose will be determined by the efficacy of the particular inventive TCR material and the condition of the animal (e.g., human), as well as the body weight of the animal (e.g., human) to be treated.

Many assays for determining an administered dose are known in the art. For purposes of the invention, an assay, which comprises comparing the extent to which target cells are lysed or IFN-γ is secreted by T cells expressing the inventive TCR, polypeptide, or protein upon administration of a given dose of such T cells to a mammal among a set of mammals of which each is given a different dose of the T cells, could be used to determine a starting dose to be administered to a mammal. The extent to which target cells are lysed or IFN-γ is secreted upon administration of a certain dose can be assayed by methods known in the art.

The dose of the inventive TCR material also will be determined by the existence, nature and extent of any adverse side effects that might accompany the administration of a particular inventive TCR material. Typically, the attending physician will decide the dosage of the inventive TCR material with which to treat each individual patient, taking into consideration a variety of factors, such as age, body weight, general health, diet, sex, inventive TCR material to be administered, route of administration, and the severity of the cancer being treated. In an aspect in which the inventive TCR material is a population of cells, the number of cells administered per infusion may vary, e.g., from about 1×10⁶ to about 1×10¹² cells or more. In certain aspects, fewer than 1×10⁶ cells may be administered.

One of ordinary skill in the art will readily appreciate that the inventive TCR materials of the invention can be modified in any number of ways, such that the therapeutic or prophylactic efficacy of the inventive TCR materials is increased through the modification. For instance, the inventive TCR materials can be conjugated either directly or indirectly through a bridge to a chemotherapeutic agent. The practice of conjugating compounds to a chemotherapeutic agent is known in the art. One of ordinary skill in the art recognizes that sites on the inventive TCR materials, which are not necessary for the function of the inventive TCR materials, are suitable sites for attaching a bridge and/or a chemotherapeutic agent, provided that the bridge and/or chemotherapeutic agent, once attached to the inventive TCR materials, do(es) not interfere with the function of the inventive TCR materials, i.e., the ability to bind to Q61K RAS or to detect, treat, or prevent cancer.

It is contemplated that the inventive pharmaceutical compositions, TCRs, polypeptides, proteins, nucleic acids, recombinant expression vectors, host cells, and populations of cells can be used in methods of treating or preventing cancer. Without being bound to a particular theory, the inventive TCRs are believed to bind specifically to Q61K RAS, such that the TCR (or related inventive polypeptide or protein), when expressed by a cell, is able to mediate an immune response against a target cell expressing Q61K RAS. In this regard, an aspect of the invention provides a method of treating or preventing cancer in a mammal, comprising administering to the mammal any of the pharmaceutical compositions, TCRs, polypeptides, or proteins described herein, any nucleic acid or recombinant expression vector comprising a nucleotide sequence encoding any of the TCRs, polypeptides, proteins described herein, or any host cell or population of cells comprising a recombinant vector which encodes any of the TCRs, polypeptides, or proteins described herein, in an amount effective to treat or prevent cancer in the mammal.

An aspect of the invention provides a method of inducing an immune response against a cancer in a mammal, comprising administering to the mammal any of the pharmaceutical compositions. TCRs, polypeptides, or proteins described herein, any nucleic acid or recombinant expression vector comprising a nucleotide sequence encoding any of the TCRs, polypeptides, or proteins described herein, or any host cell or population of cells comprising a recombinant vector which encodes any of the TCRs, polypeptides, or proteins described herein, in an amount effective to induce an immune response against the cancer in the mammal.

An aspect of the invention provides any of the pharmaceutical compositions, TCRs, polypeptides, or proteins described herein, any nucleic acid or recombinant expression vector comprising a nucleotide sequence encoding any of the TCRs, polypeptides, proteins described herein, or any host cell or population of cells comprising a recombinant vector which encodes any of the TCRs, polypeptides, or proteins described herein, for use in the treatment or prevention of cancer in a mammal.

An aspect of the invention provides any of the pharmaceutical compositions, TCRs, polypeptides, or proteins described herein, any nucleic acid or recombinant expression vector comprising a nucleotide sequence encoding any of the TCRs, polypeptides, or proteins described herein, or any host cell or population of cells comprising a recombinant vector which encodes any of the TCRs, polypeptides, or proteins described herein, for use in inducing an immune response against a cancer in a mammal.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of cancer in a mammal. Furthermore, the treatment or prevention provided by the inventive method can include treatment or prevention of one or more conditions or symptoms of the cancer being treated or prevented. For example, treatment or prevention can include promoting the regression of a tumor. Also, for purposes herein, “prevention” can encompass delaying the onset of the cancer, or a symptom or condition thereof. Alternatively or additionally, “prevention” may encompass preventing or delaying the recurrence of cancer, or a symptom or condition thereof.

Also provided is a method of detecting the presence of cancer in a mammal. The method comprises (i) contacting a sample comprising one or more cells from the mammal with any of the inventive TCRs, polypeptides, proteins, nucleic acids, recombinant expression vectors, host cells, populations of cells, or pharmaceutical compositions described herein, thereby forming a complex, and (ii) detecting the complex, wherein detection of the complex is indicative of the presence of cancer in the mammal.

With respect to the inventive method of detecting cancer in a mammal, the sample of cells can be a sample comprising whole cells, lysates thereof, or a fraction of the whole cell lysates, e.g., a nuclear or cytoplasmic fraction, a whole protein fraction, or a nucleic acid fraction.

For purposes of the inventive method of detecting cancer, the contacting can take place in vitro or in vivo with respect to the mammal. Preferably, the contacting is in vitro.

Also, detection of the complex can occur through any number of ways known in the art. For instance, the inventive TCRs, polypeptides, proteins, nucleic acids, recombinant expression vectors, host cells, or populations of cells, described herein, can be labeled with a detectable label such as, for instance, a radioisotope, a fluorophore (e.g., fluorescein isothiocyanate (FITC), phycoerythrin (PE)), an enzyme (e.g., alkaline phosphatase, horseradish peroxidase), and element particles (e.g., gold particles).

For purposes of the inventive methods, wherein host cells or populations of cells are administered, the cells can be cells that are allogeneic or autologous to the mammal. Preferably, the cells are autologous to the mammal.

With respect to the inventive methods, the cancer can be any cancer, including any of acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vagina, cancer of the vulva, chronic lymphocytic leukemia, chronic myeloid cancer, colon cancer, colorectal cancer, endometrial cancer, esophageal cancer, uterine cervical cancer, gastrointestinal carcinoid tumor, glioma, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, cancer of the oropharynx, ovarian cancer, cancer of the penis, pancreatic cancer, peritoneum, omentum, and mesentery cancer, pharynx cancer, prostate cancer, rectal cancer, renal cancer, skin cancer, small intestine cancer, soft tissue cancer, stomach cancer, testicular cancer, thyroid cancer, cancer of the uterus, ureter cancer, and urinary bladder cancer. A preferred cancer is melanoma. In an aspect of the invention, the cancer expresses a mutated human RAS amino acid sequence with a substitution of glutamine at position 61 with lysine, wherein the mutated human RAS amino acid sequence is a mutated human KRAS, a mutated human HRAS, or a mutated human NRAS amino acid sequence, and wherein position 61 is defined by reference to the WT human KRAS, WT human HRAS, or WT human NRAS protein, respectively. The mutated human KRAS, mutated human HRAS, and mutated human NRAS expressed by the cancer may be as described herein with respect to other aspects of the invention.

The mammal referred to in the inventive methods can be any mammal. As used herein, the term “mammal” refers to any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

The following examples further illustrate the invention but, of course, should not be construed as in any way limiting its scope.

### EXAMPLE 1

This example demonstrates the identification of TIL from melanoma patient 3795 having reactivity against one of four mutant antigens expressed by melanoma patient 3795.

TILs from tumor fragments from melanoma patient 3795 were independently screened for reactivity against Q61K RAS. Nonsynonymous mutations were identified in tumor fragments (numbered F1-F2 and F4-F8) from patient 3795 by whole exome and RNA-sequnce analysis. The nonsynonymous mutations identified were used to generate 11 tandem minigene (TMG) constructs (TMG1-TMG11) and three peptide pools (PP) (PP1-PP3), as described previously (Lu et al., Clin. Cancer Res., 20: 3401-3410 (2014)).

TIL from tumor fragments (numbered F1-F2 and F4-F8) from patient 3795 (effector cells) were independently co-cultured with autologous DC (target cells). RX1 was a sample of the TIL product which had been administered to Patient 3795 for treatment. RX1 comprised a combination of TIL from tumor fragments F1, F2, F4, and F5. RX1 served as alternative effector cells. The DC were (i) independently transfected with mRNA encoding one of the 11 TMG constructs (TMG1-TMG11) or (ii) independently pulsed with one of PP1-PP3. Effector cells (i) co-cultured with DC treated with DMSO, (ii) cultured alone (TIL only), or (iii) cultured with DC transfected with a TMG constructing encoding irrelevant mutations served as negative controls. Effector cells treated with anti-CD3 antibody (OKT3) served as a positive control.

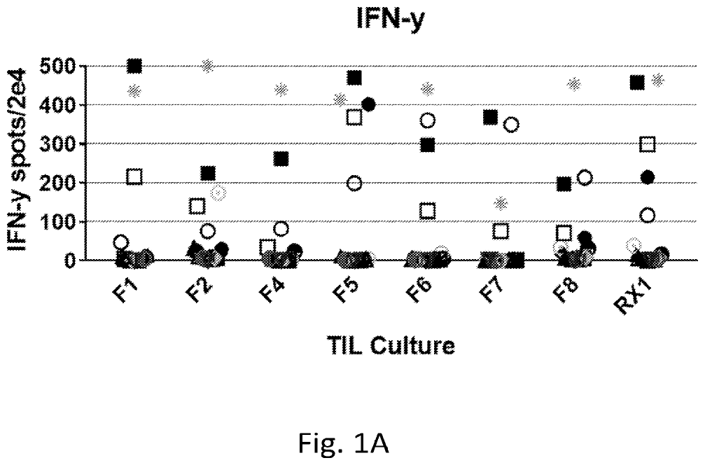

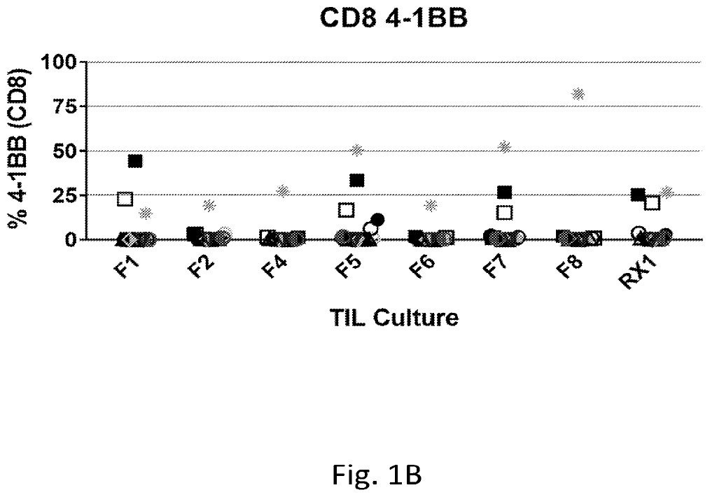

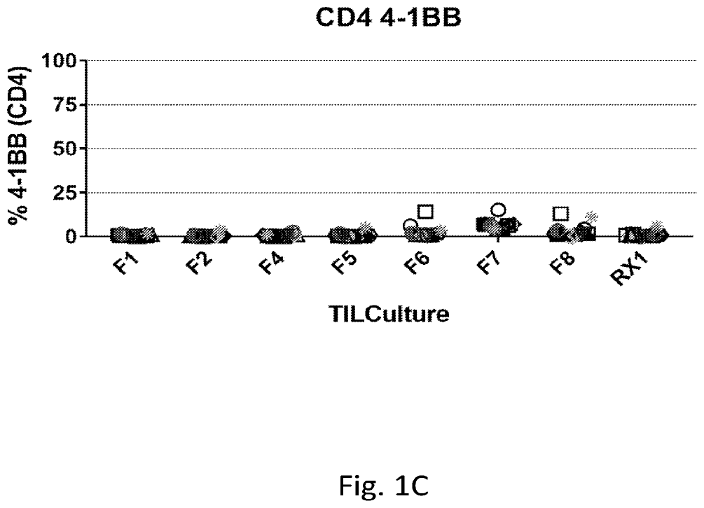

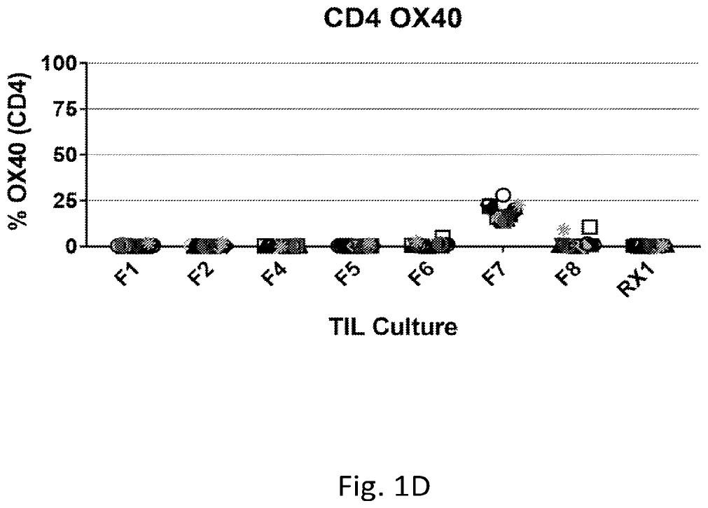

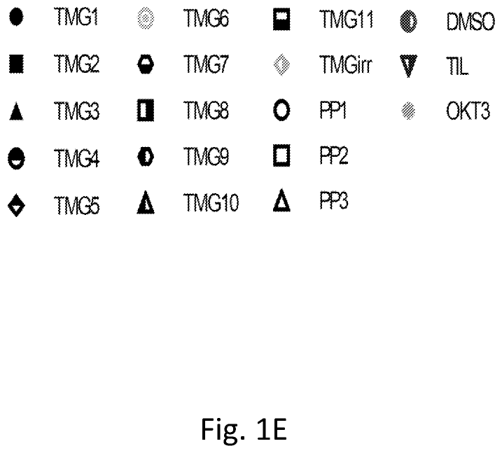

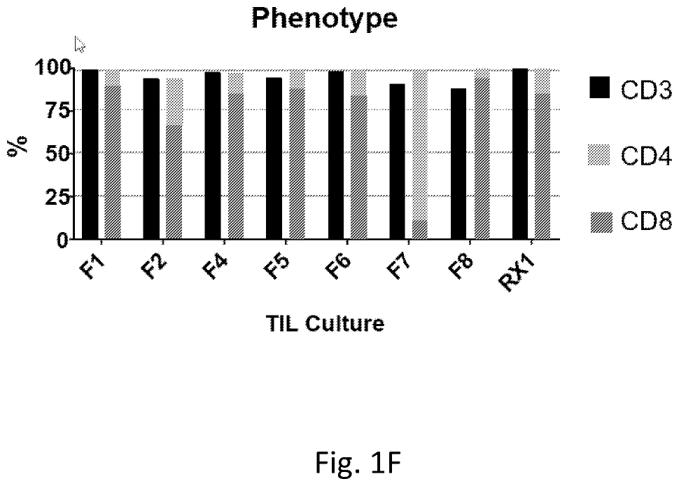

Reactivity was tested by IFNγ-secretion using ELISpot assay (FIG. 1A) and by measuring the expression of one or both of 4-1BB and OX40 by flow cytometry assay gated on (i) CD3⁺/CD4⁺ cells (FIGS. 1C-1D) or (ii) CD3⁺/CD8⁺ cells (FIG. 1B). Reactive cells were observed following co-culture of the TIL from multiple tumor fragments with DCs which had been pulsed with PP1 or PP2 and following co-culture of the TIL from multiple tumor fragments with DCs which had been transfected with mRNA encoding TMG1 or TMG2. F2 was the only tumor fragment with TIL which were reactive to DCs which had been transfected with mRNA encoding TMG6. The number of positive ELISPOT wells detected following co-culture of TIL from tumor fragment F5 with target cells and controls is shown in Table 5.

TABLE 5

| | Number of | | Number of |
|---|---|---|---|
| Target | positive | Target | positive |
| cell | ELISPOT wells | cell | ELISPOT wells |
| TMG1 | 402 | TMG10 | 2 |
| TMG2 | 470 | TMG11 | 2 |
| TMG3 | 12 | TMGirr | 3 |
| TMG4 | 3 | PP1 | 199 |
| TMG5 | 4 | PP2 | 369 |
| TMG6 | 5 | PP3 | 4 |
| TMG7 | 0 | DMSO | 3 |
| TMG8 | 0 | TIL alone | 0 |
| TMG9 | 1 | OKT3 | 414 |

The phenotype of the TIL (cultured alone) from each tumor fragment was analyzed. The reactivity appeared to be predominantly CD8⁺ (FIG. 1F).

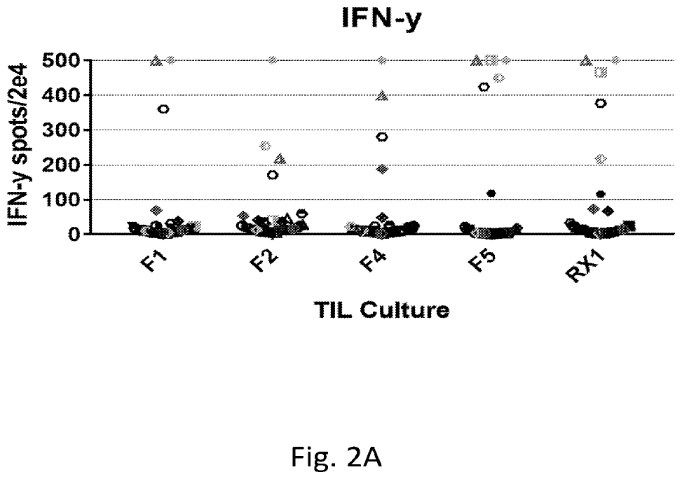

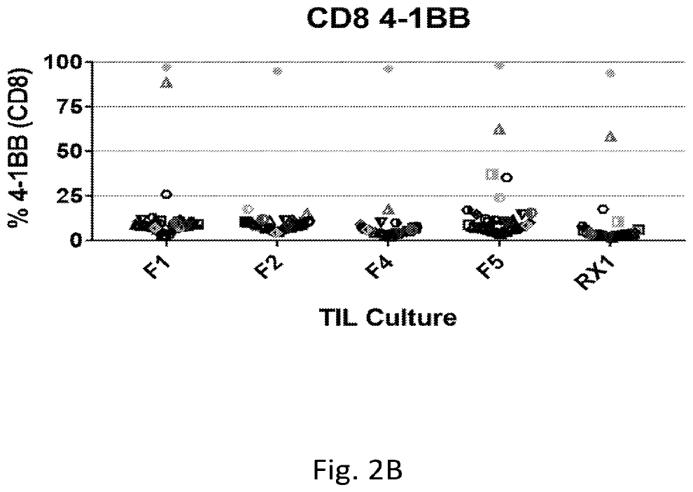

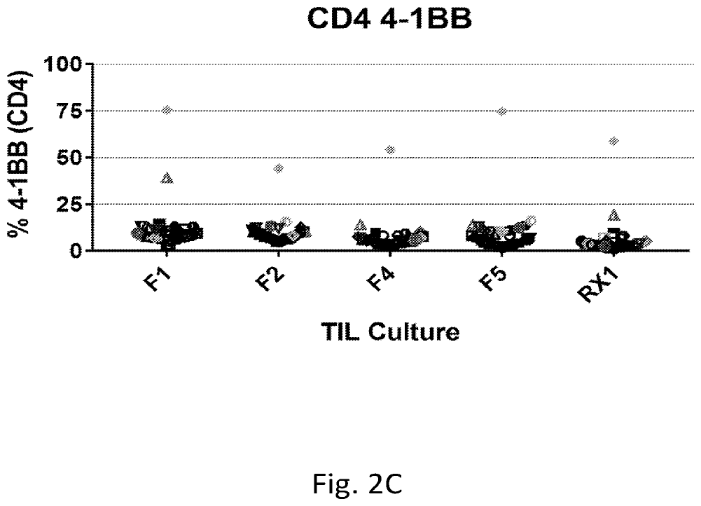

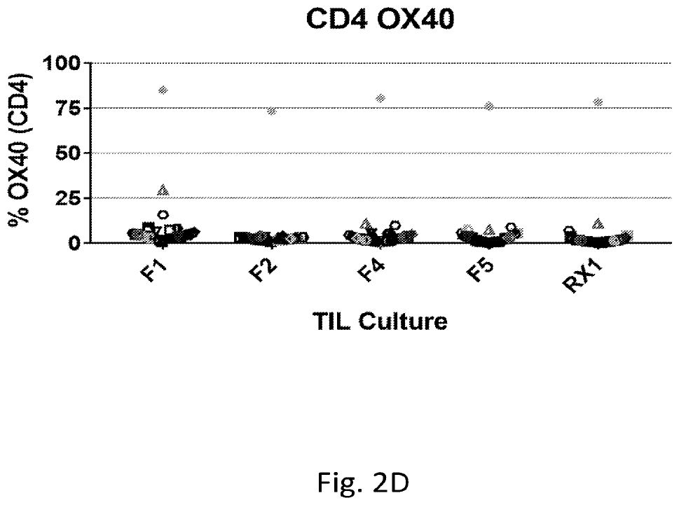

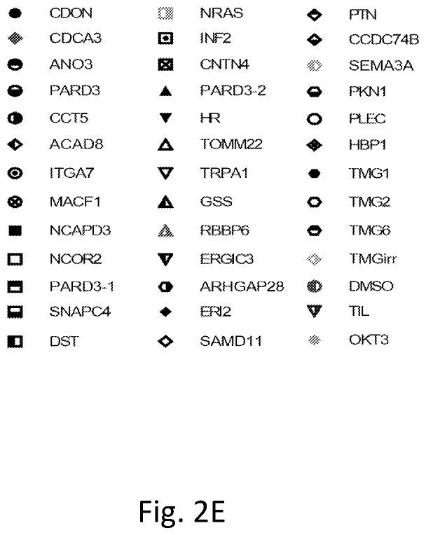

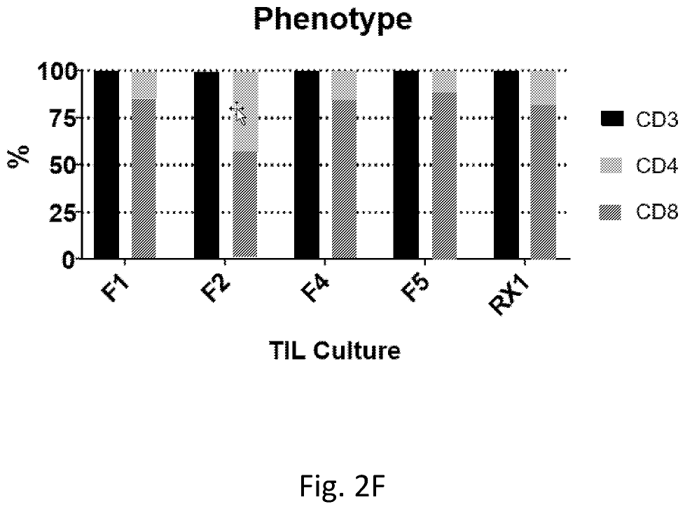

Because reactivity was observed with TIL from multiple tumor fragments, further reactivity studies were carried out. In these further reactivity studies, autologous DCs were independently pulsed with individual peptides from PP1 and PP2 (shown in FIG. 2E). These pulsed DCs (target cells) were co-cultured with effector cells. The effector cells were the TIL of RX1 or the TIL of only one of the tumor fragments that comprised RX1 (namely, F1, F2, F4, or F5).

Reactivity was tested by IFNγ-secretion using ELISpot assay (FIG. 2A) and by measuring the expression of one or both of 4-1BB and OX40 by flow cytometry assay gated on (i) CD3⁺/CD4⁺ cells (FIGS. 2C-2D) or (ii) CD3⁺/CD8⁺ cells (FIG. 2B). The number of positive ELISPOT wells detected following co-culture of TIL from tumor fragment F5 with target cells and controls is shown in Table 6. Reactivity of TIL from tumor fragments F1, F2, F4, F5, and RX1 was observed against four mutant antigens (CDCA3, NRAS, RBBP6, and SEMA3A) shown in Table 7. The reactivity shown in FIGS. 2A-2D is summarized in Table 8.

TABLE 6

| | Number of | | Number of |
|---|---|---|---|
| | positive | | positive |
| Peptide | ELISPOT wells | Peptide | ELISPOT wells |
| CDON | 8 | TRPA1 | 3 |
| CDCA3 | 7 | GSS | 0 |
| ANO3 | 0 | RBBP6 | 353 |
| PARD3 | 2 | ERGIC3 | 4 |
| CCT5 | 21 | ARHGAP28 | 3 |
| ACAD8 | 1 | ERI2 | 2 |
| ITGA7 | 4 | SAMD11 | 5 |
| MACF1 | 3 | PTN | 16 |
| NCAPD3 | 4 | CCDC748 | 4 |
| NCOR2 | 4 | SEMA3A | 450 |
| PARD3-1 | 4 | PKN1 | 10 |
| SNAPC4 | 3 | PLEC | 3 |
| DST | 2 | HBP1 | 18 |
| NRAS | 500 | TMG1 | 118 |
| INF2 | 4 | TMG2 | 424 |
| CNTN4 | 14 | TMG6 | 6 |
| PARD3-2 | 13 | TMGirr | 4 |
| HR | 5 | DMSO | 3 |
| TOMM22 | 15 | TIL | 5 |
| — | — | OKT | 42 |

TABLE 7

| GENE | SEQUENCE | SEQ ID NO: |
|---|---|---|
| CDCA3 | NSPGTLTLRQGKQPSPLSENVSELK | 33 |
| NRAS | ETCLLDILDTAGKEEYSAMRDQYMR | 34 |
| RBBP6 | PNKRNVPQGETECEYFNRYREVPPP | 35 |
| SEMA3A | AYNQTHLYACGTRAFHPICTYIEIG | 36 |

TABLE 8

| | | CD8⁺ | CD4⁺ | CD4⁺ |
|---|---|---|---|---|
| GENE | IFN-y ELISPOT | 4-1BB | 4-1BB | OX40 |
| CDCA3 | F1, F4, RX1 | None | None | None |
| NRAS | F5, RX1 | F5, RX1 | None | None |
| RBBP6 | F1, F2, F4, | F1, F4, F5, | F1, F4, | F1, |
| | F5, RX1 | RX1 | RX1 | RX1 |
| SEMA3A | F2, F5, RX1 | F2, F5 | None | None |

The phenotype of the TIL (cultured alone) from each tumor fragment was analyzed. The reactivity appeared to be predominantly CD8⁺ (FIG. 2F).

### EXAMPLE 2

This example demonstrates the avidity of the TIL from tumor fragment F5 which were identified as reactive against Q61K RAS in Example 1.

The avidity of the TIL from tumor fragment F5 which were identified as reactive against Q61K RAS in Example 1 was tested as follows. The TILs were co-cultured with autologous DC pulsed with various concentrations of the mutated NRAS peptides shown in Table 9. The peptide 10-mer NRAS-R corresponds to NRAS Q61R, which is another common mutation at this position.

TABLE 9

| Peptide name | Sequence | SEQ ID NO: |
|---|---|---|
| 10-mer NRAS-0 | ILDTAGKEEY | 37 |
| 11-mer NRAS-1 | DILDTAGKEEY | 38 |
| 12-mer NRAS-2 | LDILDTAGKEEY | 39 |
| 13-mer NRAS-3 | LLDILDTAGKEEY | 40 |
| 10-mer NRAS-R | ILDTAGREEY | 41 |

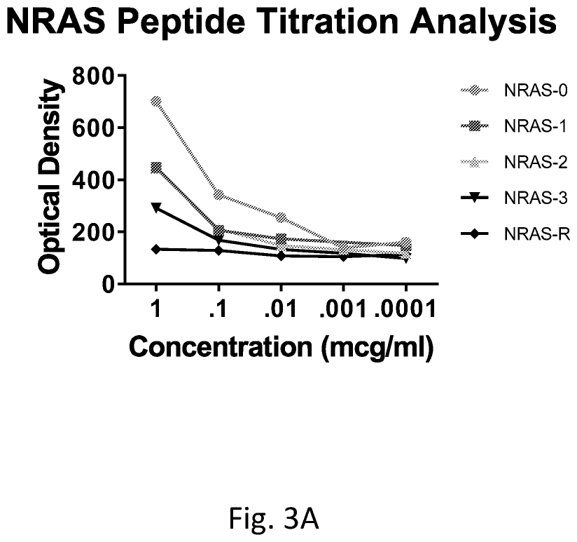

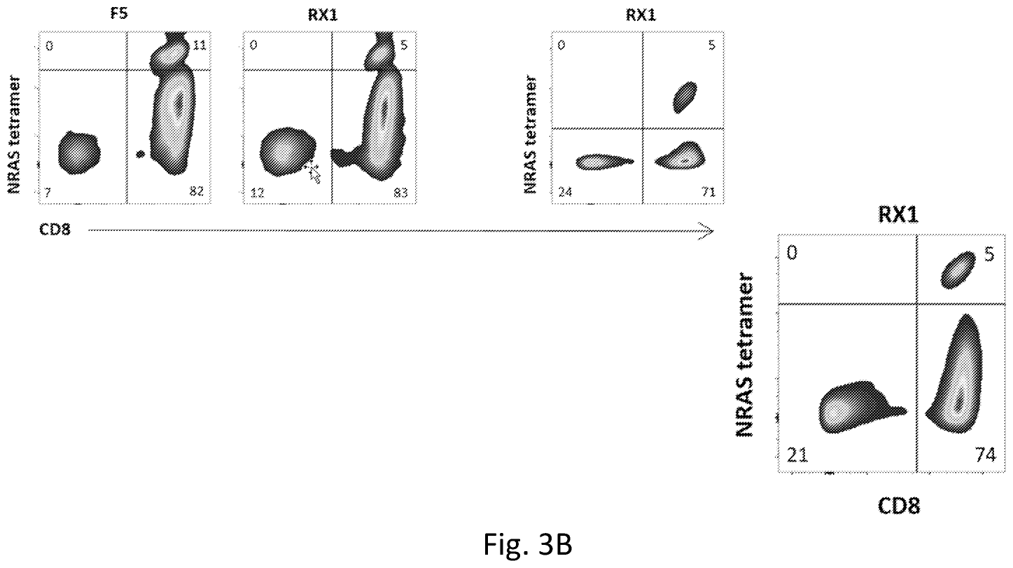

Reactivity was tested by evaluating the response to the peptide variants, evaluated by determining the optical density of the developing reagent TMB (tetramethylbenzidine) generated in a standard ELISA co-culture assay. The results are shown in FIG. 3A. The results demonstrated that the highest avidity was achieved with the 10-mer peptide ILDTAGKEEY (SEQ ID NO: 37). The TIL did not recognize the peptide ILDTAGREEY (SEQ ID NO: 41).

### EXAMPLE 3

This example demonstrates that the TIL from tumor fragment F5 recognize Q61K RAS presented by HLA-A*01:01.

COS cells, a highly transfectable monkey kidney tumor cell line (target cells), were independently transfected with mRNA encoding one of the HLA restriction elements shown in Table 10. The target cells were also transfected with mRNA encoding TMG1 or no TMG. TIL from tumor fragment F5 and RX1 (effector cells) were independently co-cultured with the target cells. Reactivity was tested by IFNγ-secretion using ELISpot assay. The results are shown in Table 10. The values in Table 10 are IFN-gamma (pg/ml).

TABLE 10

| | Effector | | | |
|---|---|---|---|---|
| | cells | Fragment F5 | RX1 | RX1 |
| | Target cells | TMG 1 | TMG 1 | No TMG |
| HLA | A*01:01 | >2000 | >2000 | 94 |
| restriction | A*02:01 | <30 | <30 | <30 |
| element | B*08:01 | <30 | <30 | <30 |
| | B*27:01 | <30 | <30 | <30 |
| | C*07:01 | <30 | <30 | <30 |
| | No HLA | <30 | <30 | <30 |

### EXAMPLE 4

This example demonstrates the isolation of anti-Q61K RAS TCR from the TIL which were identified as reactive to Q61K RAS presented by HLA-A*01:01 in Example 1-3.

The minimal epitope was identified as the 10-mer peptide ILDTAGKEEY (SEQ ID NO: 37) in Example 2. A tetramer was synthesized with the 10-mer.

Screening for the presence of T cells with TCRs with desired specificity was performed with p-MHC tetramer staining. TCR tetramers were identified. Results are presented in FIG. 3B. Tetramer-bound T cell population was isolated by staining cells with PE-conjugated p-MHC tetramer followed by enrichment.

Paired single cell TCR sequencing was performed to identify paired TCRα and TCRβ chain sequences (TCRα (TCRAV) and TCRβ (TCRBV)). Sequencing was performed on bulk lymphocytes and/or T cells enriched for tetramer-bound populations as described in the paragraph above. The TCR alpha and beta chains (TRAV19/AJ39 and TRBV6-1/BJ1-1) were identified.

The amino acid sequences of the alpha and beta chain variable regions are shown in Table 11. The CDRs are underlined. The N-terminal signal peptides are in bold font.

TABLE 11

| TCR chain | Amino acid sequence |
|---|---|
| Variable α | AQKVTQAQTEISVVEKEDVTLDCVYETRDTTYYLF |
| (Predicted | WYKQPPSGELVFLIRRNSFDEQNEISGRYSWNFQKS |
| sequence | TSSFNFTITASQVVDSAVYFCALSESHNNAGNMLTF |
| without N- | GGGTRLMVKP (SEQ ID NO: 7) |
| terminal signal | |
| peptide) | |
| Variable ß | NAGVTQTPKFQVLKTGQSMTLQCAQDMNHNSMY |
| (Predicted | WYRQDPGMGLRLIYYSASEGTTDKGEVPNGYNVS |
| sequence | RLNKREFSLRLESAAPSQTSVYFCAASQNTEAFFGQ |
| without N- | GTRLTVV (SEQ ID NO: 8) |
| terminal signal | |
| peptide) | |
| Variable α | MLTASLLRAVIASICVVSSMAQKVTQAQTEISVVE |
| (With N- | KEDVTLDCVYETRDTTYYLFWYKQPPSGELVFLIRR |
| terminal signal | NSFDEQNEISGRYSWNFQKSTSSFNFTITASQVVDSA |
| peptide) | VYFCALSESHNNAGNMLTFGGGTRLMVKP (SEQ |
| | ID NO: 9) |
| Variable ß | MSIGLLCCVAFSLLWASPVNAGVTQTPKFQVLKT |
| (With N- | GQSMTLQCAQDMNHNSMYWYRQDPGMGLRLIYY |
| terminal signal | SASEGTTDKGEVPNGYNVSRLNKREFSLRLESAAPS |
| peptide) | QTSVYFCAASQNTEAFFGQGTRLTVV (SEQ ID NO: |
| | 10) |

### EXAMPLE 5

This example demonstrates the construction of a retroviral vector encoding the TCR of Example 4.

Nucleotide sequences encoding the variable regions of the α and β chains of the TCR of Example 4 were obtained and codon-optimized (SEQ ID NOs: 42-43, respectively). The TCRβ VDJ regions were fused to the mouse TCRβ constant chain. The TCRα VJ regions were fused to the mouse TCRα constant chain. Without being bound to a particular theory or mechanism, it is believed that replacing the constant regions of the human TCRα and TCRβ chains with the corresponding murine constant regions improves TCR expression and functionality (Cohen et al., Cancer Res., 66(17): 8878-86 (2006)).

In addition, the murine TCRα and TCRβ constant chains were cysteine-modified. Transmembrane hydrophobic mutations were introduced into the murine TCRα constant chain. Without being bound to a particular theory or mechanism, it is believed that these modifications result in preferential pairing of the introduced TCR chains and enhanced TCR surface expression and functionality (Cohen et al., Cancer Res., 67(8):3898-903 (2007); Haga-Friedman et al., J. Immu., 188: 5538-5546 (2012)). The full length α and β chains of each of the four TCRs, including these modifications to the constant region, are shown in Table 12. In Table 12, the CDRs are underlined, and the modified amino acid residues of the constant region are underlined and in bold.

TABLE 12

| TCR chain | Sequence |
|---|---|
| Cys-substituted, | MLTASLLRAVIASICVVSSMAQKVTQAQTEISVVEKEDVTL |
| LVL-modified TCR | DCVYETRDTTYYLFWYKQPPSGELVFLIRRNSFDEQNEISGR |
| α chain with N- | YSWNFQKSTSSFNFTITASQVVDSAVYFCALSESHNNAGNM |
| terminal signal | LTFGGGTRLMVKPNIQNPEPAVYQLKDPRSQDSTLCLFTDF |
| peptide | DSQINVPKTMESGTFITDKCVLDMKAMDSKSNGAIAWSNQ |
| | TSFTCQDIFKETNATYPSSDVPCDATLTEKSFETDMNLNFQN |
| | LLVIVLRILLLKVAGFNLLMTLRLWSS (SEQ ID NO: 29) |
| Cys-substituted, | MSIGLLCCVAFSLLWASPVNAGVTQTPKFQVLKTGQSMTLQ |
| LVL-modified TCR | CAQDMNHNSMYWYRQDPGMGLRLIYYSASEGTTDKGEVP |
| ß chain with N- | NGYNVSRLNKREFSLRLESAAPSQTSVYFCAASQNTEAFFG |
| terminal signal | QGTRLTVVEDLRNVTPPKVSLFEPSKAEIANKQKATLVCLA |
| peptide | RGFFPDHVELSWWVNGKEVHSGVCTDPQAYKESNYSYCLS |
| | SRLRVSATFWHNPRNHFRCQVQFHGLSEEDKWPEGSPKPVT |
| | QNISAEAWGRADCGITSASYQQGVLSATILYEILLGKATLYA |
| | VLVSTLVVMAMVKRKNS (SEQ ID NO: 30) |
| Cys-substituted, | AQKVTQAQTEISVVEKEDVTLDCVYETRDTTYYLFWYKQP |
| LVL-modified TCR | PSGELVFLIRRNSFDEQNEISGRYSWNFQKSTSSFNFTITASQ |
| α chain predicted | VVDSAVYFCALSESHNNAGNMLTFGGGTRLMVKPNIQNPE |
| sequence without N- | PAVYQLKDPRSQDSTLCLFTDFDSQINVPKTMESGTFITDKC |
| terminal signal | VLDMKAMDSKSNGAIAWSNQTSFTCQDIFKETNATYPSSDV |
| peptide | PCDATLTEKSFETDMNLNFQNLLVIVLRILLLKVAGFNLLMT |
| | LRLWSS (SEQ ID NO: 31) |
| Cys-substituted, | NAGVTQTPKFQVLKTGQSMTLQCAQDMNHNSMYWYRQDP |
| LVL-modified TCR | GMGLRLIYYSASEGTTDKGEVPNGYNVSRLNKREFSLRLES |
| ß chain predicted | AAPSQTSVYFCAASQNTEAFFGQGTRLTVVEDLRNVTPPKV |
| sequence without N- | SLFEPSKAEIANKQKATLVCLARGFFPDHVELSWWVNGKEV |
| terminal signal | HSGVCTDPQAYKESNYSYCLSSRLRVSATFWHNPRNHFRCQ |
| peptide | VQFHGLSEEDKWPEGSPKPVTQNISAEAWGRADCGITSASY |
| | QQGVLSATILYEILLGKATLYAVLVSTLVVMAMVKRKNS |
| | (SEQ ID NO: 32) |

Nucleotide sequences encoding the variable regions of the α and β chains of the TCR of Table 12 were independently cloned into a MSGV1-based retroviral vector with the following expression cassette configuration: 5′NcoI-VDJβ-mCβ-Furin/SerGly/P2A-VJα-mCα-EcoRI3′.

The TCRβ and TCRα chains were separated by a Furin Ser/Gly P2A linker peptide (SEQ ID NO: 45). Without being bound to a particular theory or mechanism, it is believed that the linker peptide provides comparable expression efficiency of the two chains (Szymczak et al., Nat. Biotechnol., 22(5):589-94 (2004)). The TCR expression cassette of the retroviral vector encoded, from 5′ to 3′, the TCRβ and TCRα chains separated by the linker peptide. The amino acid sequence encoded by the TCR expression cassette is shown in Table 13. In Table 13, the CDRs are underlined, the constant region is italicized, and the linker peptide is shown in bold.

TABLE 13

| TCR Name | Amino acid sequence encoded by TCR Expression Cassette |
|---|---|
| 3795 | MSIGLLCCVAFSLLWASPVNAGVTQTPKFQVLKTGQSMTLQCAQ |
| | DMNHNSMYWYRQDPGMGLRLIYYSASEGTTDKGEVPNGYNVSR |
| | LNKREFSLRLESAAPSQTSVYFCAASQNTEAFFGQGTRLTVVEDLR |
| | NVTPPKVSLFEPSKAELANKQKATLVCLARGFFPDHVELSWWVNGKEV |
| | HSGVCTDPQAYKESNYSYCLSSRLRVSATFWHNPRNHFRCQVQFHGLS |
| | EEDKWPEGSPKPVTQNISAEAWGRADCGITSASYQQGVLSATILYEILL |
| | GKATLYAVLVSTLVVMAMVKRKNSRAKRSGSGATNFSLLKQAGDV |
| | EENPGPMLTASLLRAVIASICVVSSMAQKVTQAQTEISVVEKEDV |
| | TLDCVYETRDTTYYLFWYKQPPSGELVFLIRRNSFDEQNEISGRYS |
| | WNFQKSTSSFNFTITASQVVDSAVYFCALSESHNNAGNMLTFGGG |
| | TRLMVKPNIQNPEPAVYQLKDPRSQDSTLCLFTDFDSQINVPKTMESG |
| | TFITDKCVLDMKAMDSKSNGAIAWSNQTSFTCQDIFKETNATYPSSDV |
| | PCDATLTEKSFETDMNLNFQNLLVIVLRILLLKVAGENLLMTLRLWSS |
| | (SEQ ID NO: 46) |

### EXAMPLE 6

This example demonstrates the avidity of the TCR of Example 4.

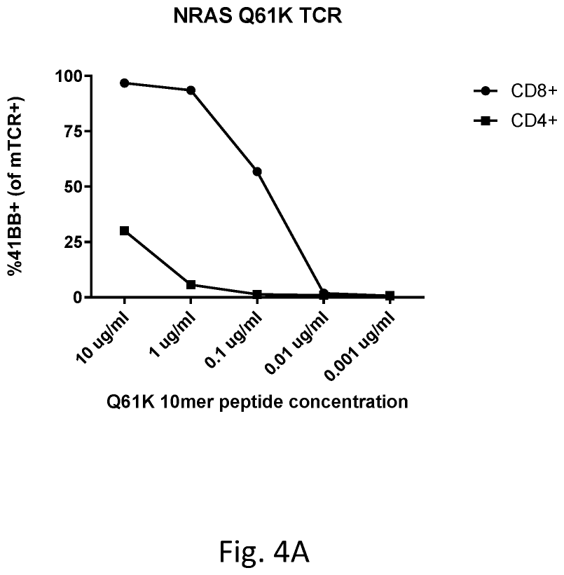

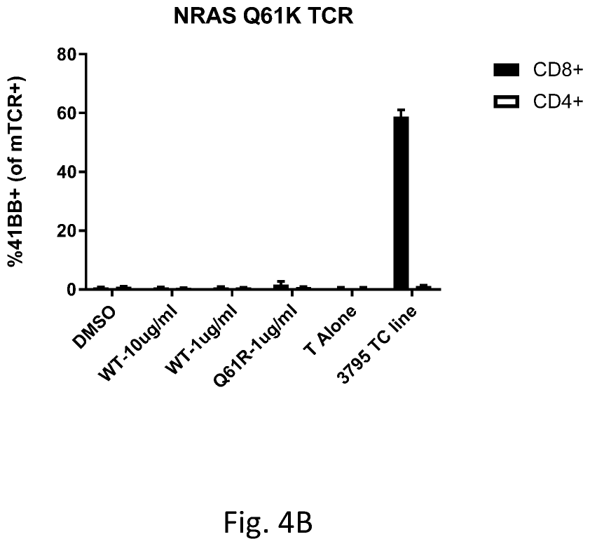

Effector cells were healthy donor PBMC sorted for CD4⁺ or CD8⁺ expression and transduced with the retroviral vector of Example 5. Target cells were autologous DCs (Patient 3795) pulsed with various concentrations of the 10-mer ILDTAGKEEY (SEQ ID NO: 37) peptide. 30,000 effector cells were co-cultured overnight with 60,000 target cells. 4-1BB upregulation by the effector cells was measured approximately 16 hours after co-culture via flow cytometry. The results are shown in FIG. 4A. As shown in FIG. 4A, the CD8⁺ effector cells recognized the target cells at peptide concentrations greater than 0.01 μg/ml.

### EXAMPLE 7

This example demonstrates that the TCR of Example 4 specifically recognizes an autologous tumor cell line from Patient 3795.

Target cells were autologous tumor cell line (Patient 3795) or autologous DCs (Patient 3795) pulsed with 1 or 10 μg/ml of wild-type (WT) peptide ILDTAGQEEY (SEQ ID NO: 44) or 1 μg/ml of 10-mer ILDTAGQEEY (SEQ ID NO: 41) (NRAS-R). DCs treated with DMSO and T cells cultured alone served as controls. Effector cells and co-culture were as described in Example 6.

The results are shown in FIG. 4B. As shown in FIG. 4B, the CD8⁺ effector cells specifically recognized the autologous tumor cell line.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An isolated or purified T-cell receptor (TCR) having antigenic specificity for a mutated human RAS amino acid sequence with a substitution of glutamine at position 61 with lysine,
wherein the mutated human RAS amino acid sequence is a mutated human Kirsten rat sarcoma viral oncogene homolog (KRAS), a mutated human Harvey rat sarcoma viral oncogene homolog (HRAS), or a mutated human Neuroblastoma rat sarcoma viral oncogene homolog (NRAS) amino acid sequence, and
wherein position 61 is defined by reference to the wild-type human KRAS, wild-type human HRAS, or wild-type human NRAS protein, respectively.

2. The isolated or purified TCR of claim 1 comprising the amino acid sequences of:
(a) all of SEQ ID NOs: 1-3,
(b) all of SEQ ID NOs: 4-6, or
(c) all of SEQ ID NOs: 1-6.

3-7. (canceled)

8. The TCR according to claim 1, comprising the amino acid sequence(s) of:
(1) SEQ ID NO: 7,
(2) SEQ ID NO: 8,
(3) SEQ ID NO: 9,
(4) SEQ ID NO: 10,
(5) both of SEQ ID NOs: 7 and 8, or
(6) both of SEQ ID NOs: 9 and 10.

9. The TCR of claim 1, further comprising:
(a) the amino acid sequence of SEQ ID NO: 19, wherein:
(i) X at position 48 of SEQ ID NO: 19 is Thr or Cys;
(ii) X at position 112 of SEQ ID NO: 19 is Ser, Ala, Val, Leu, Ile, Pro, Phe, Met, or Trp;
(iii) X at position 114 of SEQ ID NO: 19 is Met, Ala, Val, Leu, Ile, Pro, Phe, or Trp; and
(iv) X at position 115 of SEQ ID NO: 19 is Gly, Ala, Val, Leu, Ile, Pro, Phe, Met, or Trp;
(b) the amino acid sequence of SEQ ID NO: 20, wherein X at position 57 of SEQ ID NO: 20 is Ser or Cys; or
(c) both (a) and (b).

10. (canceled)

11. An isolated or purified polypeptide comprising a functional portion of the TCR of claim 1, wherein the functional portion comprises the amino acid sequences of:
(a) all of SEQ ID NOs: 1-3,
(b) all of SEQ ID NOs: 4-6, or
(c) all of SEQ ID NOs: 1-6.

12. The isolated or purified polypeptide according to claim 11, wherein the functional portion comprises the amino acid sequence(s) of:
(1) SEQ ID NO: 7,
(2) SEQ ID NO: 8,
(3) SEQ ID NO: 9,
(4) SEQ ID NO: 10,
(5) both of SEQ ID NOs: 7 and 8, or
(6) both of SEQ ID NOs: 9 and 10.

13. The isolated or purified polypeptide of claim 11, further comprising:
(a) the amino acid sequence of SEQ ID NO: 19, wherein:
(i) X at position 48 of SEQ ID NO: 19 is Thr or Cys;
(ii) X at position 112 of SEQ ID NO: 19 is Ser, Ala, Val, Leu, Ile, Pro, Phe, Met, or Trp;
(iii) X at position 114 of SEQ ID NO: 19 is Met, Ala, Val, Leu, Ile, Pro, Phe, or Trp; and
(iv) X at position 115 of SEQ ID NO: 19 is Gly, Ala, Val, Leu, Ile, Pro, Phe, Met, or Trp;
(b) the amino acid sequence of SEQ ID NO: 20, wherein X at position 57 of SEQ ID NO: 20 is Ser or Cys; or
(c) both (a) and (b).

14. (canceled)

15. An isolated or purified protein comprising first and second polypeptide chains, wherein:
(a) the first polypeptide chain comprising the amino acid sequences of SEQ ID NOs: 1-3;
(b) the second polypeptide chain comprising the amino acid sequences of SEQ ID NOs: 4-6; or
(c) both (a) and (b).

16. The isolated or purified protein according to claim 15, wherein:
(i) the first polypeptide chain comprises the amino acid sequence of SEQ ID NO: 7;
(ii) the second polypeptide chain comprises the amino acid sequence of SEQ ID NO: 8;
(iii) the first polypeptide chain comprises the amino acid sequence of SEQ ID NO: 9;
(iv) the second polypeptide chain comprises the amino acid sequence of SEQ ID NO: 10;
(v) both (i) and (ii); or
(vi) both (iii) and (iv).

17. The isolated or purified protein of claim 15, wherein:
(a) the first polypeptide chain further comprises the amino acid sequence of SEQ ID NO: 19, wherein:
(i) X at position 48 of SEQ ID NO: 19 is Thr or Cys;
(ii) X at position 112 of SEQ ID NO: 19 is Ser, Ala, Val, Leu, Ile, Pro, Phe, Met, or Trp;
(iii) X at position 114 of SEQ ID NO: 19 is Met, Ala, Val, Leu, Ile, Pro, Phe, or Trp; and
(iv) X at position 115 of SEQ ID NO: 19 is Gly, Ala, Val, Leu, Ile, Pro, Phe, Met, or Trp;
(b) the second polypeptide chain further comprises the amino acid sequence of SEQ ID NO: 20, wherein X at position 57 of SEQ ID NO: 20 is Ser or Cys; or
(c) both (a) and (b).

18. (canceled)

19. An isolated or purified nucleic acid comprising a nucleotide sequence encoding the TCR according to claim 1.

20. An isolated or purified nucleic acid comprising, from 5′ to 3′, a first nucleic acid sequence and a second nucleotide sequence, wherein the first and second nucleotide sequence, respectively, encode the amino sequences of SEQ ID NOs: 7 and 8; 8 and 7; 9 and 10; 10 and 9; 25 and 26; 26 and 25; 27 and 28; 28 and 27; 29 and 30; 30 and 29; 31 and 32; or 32 and 31.

21. (canceled)

22. A recombinant expression vector comprising the nucleic acid according to claim 19.

23-25. (canceled)

26. A method of producing a host cell expressing a TCR that has antigenic specificity for the peptide of ILDTAGKEEY (SEQ ID NO: 37), the method comprising contacting a cell in vitro with the recombinant expression vector according to claim 22 under conditions that allow introduction of the recombinant expression vector into the cell.

27. An isolated or purified host cell comprising the nucleic acid according to claim 19.

28-29. (canceled)

30. An isolated or purified population of cells comprising the host cell according to claim 27.

31. A method of producing a TCR, the method comprising culturing the host cell according to claim 27 so that the TCR is produced.

32. A pharmaceutical composition comprising (a) the population of cells according to claim 30 and (b) a pharmaceutically acceptable carrier.

33. A method of detecting the presence of cancer in mammal, the method comprising:
(a) contacting a sample comprising cells of the cancer with the TCR according to claim 1, thereby forming a complex; and
(b) detecting the complex,
wherein detection of the complex is indicative of the presence of cancer in the mammal.

34-40. (canceled)

41. A method of inducing an immune response against cancer in a mammal, the method comprising administering to the mammal the population of cells according to claim 30 in an amount effective to induce the immune response against cancer in the mammal.

42. A method of treating or preventing cancer in a mammal, the method comprising administering to the mammal the population of cells according to claim 30 in an amount effective to treat or prevent cancer in the mammal.


SEQUENCE LISTING (46 sequences)

SEQ ID NO: 1 - protein, 7 aa, Homo sapiens
TRDTTYY

SEQ ID NO: 2 - protein, 8 aa, Homo sapiens
RNSFDEQN

SEQ ID NO: 3 - protein, 16 aa, Homo sapiens
CALSESHNNAGNMLTF

SEQ ID NO: 4 - protein, 5 aa, Homo sapiens
MNHNS

SEQ ID NO: 5 - protein, 6 aa, Homo sapiens
SASEGT

SEQ ID NO: 6 - protein, 10 aa, Homo sapiens
CAASQNTEAF

SEQ ID NO: 7 - protein, 117 aa, Homo sapiens
AQKVTQAQTEISVVEKEDVTLDCVYETRDTTYYLFWYKQPPSGELVFLIRRNSFDEQNEISGRYSWNFQKSTSSFNFTITASQVVDSAVYFCALSESHNNAGNMLTFGGGTRLMVKP

SEQ ID NO: 8 - protein, 110 aa, Homo sapiens
NAGVTQTPKFQVLKTGQSMTLQCAQDMNHNSMYWYRQDPGMGLRLIYYSASEGTTDKGEVPNGYNVSRLNKREFSLRLESAAPSQTSVYFCAASQNTEAFFGQGTRLTVV

SEQ ID NO: 9 - protein, 137 aa, Homo sapiens
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSESHNNAGNMLTFGGGTRLMVKP

SEQ ID NO: 10 - protein, 129 aa, Homo sapiens
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCAASQNTEAFFGQGTRLTVV

SEQ ID NO: 11 - protein, 189 aa, Homo sapiens [sequence omitted]

SEQ ID NO: 12 - protein, 188 aa, Homo sapiens [sequence omitted]

SEQ ID NO: 13 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 14 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 15 - protein, 189 aa, Homo sapiens
MTEYKLVVVGAGGVGKSALTIQLIQNHFVDEYDPTIEDSYRKQVVIDGETCLLDILDTAGKEEYSAMRDQYMRTGEGFLCVFAINNTKSFEDIHHYREQIKRVKDSEDVPMVLVGNKCDLPSRTVDTKQAQDLARSYGIPFIETSAKTRQRVEDAFYTLVREIRQYRLKKISKEEKTPGCVKIKKCIIM

SEQ ID NO: 16 - protein, 188 aa, Homo sapiens
MTEYKLVVVGAGGVGKSALTIQLIQNHFVDEYDPTIEDSYRKQVVIDGETCLLDILDTAGKEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEKMSKDGKKKKKKSKTKCVIM

SEQ ID NO: 17 - protein, 189 aa, Homo sapiens
MTEYKLVVVGAGGVGKSALTIQLIQNHFVDEYDPTIEDSYRKQVVIDGETCLLDILDTAGKEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 18 - protein, 189 aa, Homo sapiens
MTEYKLVVVGAGGVGKSALTIQLIQNHFVDEYDPTIEDSYRKQVVIDGETCLLDILDTAGKEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 19 - protein, 137 aa, Artificial Sequence
  Synthetic
  MISC_FEATURE (48)..(48): X is Thr or Cys
  MISC_FEATURE (112)..(112): X is Ser, Ala, Val, Leu, Ile, Pro, Phe, Met, or Trp
  MISC_FEATURE (114)..(114): X is Met, Ala, Val, Leu, Ile, Pro, Phe, or Trp
  MISC_FEATURE (115)..(115): X is Gly, Ala, Val, Leu, Ile, Pro, Phe, Met, or Trp
NIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 20 - protein, 173 aa, Artificial Sequence
  Synthetic
  MISC_FEATURE (57)..(57): X is Ser or Cys
EDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 21 - protein, 137 aa, Artificial Sequence
  Synthetic
NIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 22 - protein, 173 aa, Artificial Sequence
  Synthetic
EDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 23 - protein, 137 aa, Mus musculus
NIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 24 - protein, 173 aa, Mus musculus
EDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 25 - protein, 274 aa, Artificial Sequence
  Synthetic
  MISC_FEATURE (185)..(185): X is Thr or Cys
  MISC_FEATURE (249)..(249): X is Ser, Ala, Val, Leu, Ile, Pro, Phe, Met, or Trp
  MISC_FEATURE (251)..(251): X is Met, Ala, Val, Leu, Ile, Pro, Phe, or Trp
  MISC_FEATURE (252)..(252): X is Gly, Ala, Val, Leu, Ile, Pro, Phe, Met, or Trp
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSESHNNAGNMLTFGGGTRLMVKPNIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 26 - protein, 302 aa, Artificial Sequence
  Synthetic
  MISC_FEATURE (186)..(186): X is Ser or Cys
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCAASQNTEAFFGQGTRLTVVEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 27 - protein, 254 aa, Artificial Sequence
  Synthetic
  MISC_FEATURE (165)..(165): X is Thr or Cys
  MISC_FEATURE (229)..(229): X is Ser, Ala, Val, Leu, Ile, Pro, Phe, Met, or Trp
  MISC_FEATURE (231)..(231): X is Met, Ala, Val, Leu, Ile, Pro, Phe, or Trp
  MISC_FEATURE (232)..(232): X is Gly, Ala, Val, Leu, Ile, Pro, Phe, Met, or Trp
AQKVTQAQTEISVVEKEDVTLDCVYETRDTTYYLFWYKQPPSGELVFLIRRNSFDEQNEISGRYSWNFQKSTSSFNFTITASQVVDSAVYFCALSESHNNAGNMLTFGGGTRLMVKPNIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 28 - protein, 283 aa, Artificial Sequence
  Synthetic
  MISC_FEATURE (167)..(167): X is Ser or Cys
NAGVTQTPKFQVLKTGQSMTLQCAQDMNHNSMYWYRQDPGMGLRLIYYSASEGTTDKGEVPNGYNVSRLNKREFSLRLESAAPSQTSVYFCAASQNTEAFFGQGTRLTVVEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 29 - protein, 274 aa, Artificial Sequence
  Synthetic
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSESHNNAGNMLTFGGGTRLMVKPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 30 - protein, 302 aa, Artificial Sequence
  Synthetic
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCAASQNTEAFFGQGTRLTVVEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 31 - protein, 254 aa, Artificial Sequence
  Synthetic
AQKVTQAQTEISVVEKEDVTLDCVYETRDTTYYLFWYKQPPSGELVFLIRRNSFDEQNEISGRYSWNFQKSTSSFNFTITASQVVDSAVYFCALSESHNNAGNMLTFGGGTRLMVKPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 32 - protein, 283 aa, Artificial Sequence
  Synthetic
NAGVTQTPKFQVLKTGQSMTLQCAQDMNHNSMYWYRQDPGMGLRLIYYSASEGTTDKGEVPNGYNVSRLNKREFSLRLESAAPSQTSVYFCAASQNTEAFFGQGTRLTVVEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 33 - protein, 25 aa, Homo sapiens
NSPGTLTLRQGKQPSPLSENVSELK

SEQ ID NO: 34 - protein, 25 aa, Homo sapiens
ETCLLDILDTAGKEEYSAMRDQYMR

SEQ ID NO: 35 - protein, 25 aa, Homo sapiens
PNKRNVPQGETECEYFNRYREVPPP

SEQ ID NO: 36 - protein, 25 aa, Homo sapiens
AYNQTHLYACGTRAFHPICTYIEIG

SEQ ID NO: 37 - protein, 10 aa, Homo sapiens
ILDTAGKEEY

SEQ ID NO: 38 - protein, 11 aa, Homo sapiens
DILDTAGKEEY

SEQ ID NO: 39 - protein, 12 aa, Homo sapiens
LDILDTAGKEEY

SEQ ID NO: 40 - protein, 13 aa, Homo sapiens
LLDILDTAGKEEY

SEQ ID NO: 41 - protein, 10 aa, Homo sapiens
ILDTAGREEY

SEQ ID NO: 42 - DNA, 411 bp, Artificial Sequence
  Synthetic
atgctgactgccagcctgttgagggcagtcatagcctccatctgtgttgtatccagcatggctcagaaggtaactcaagcgcagactgaaatttctgtggtggagaaggaggatgtgaccttggactgtgtgtatgaaacccgtgatactacttattacttattctggtacaagcaaccaccaagtggagaattggttttccttattcgtcggaactcttttgatgagcaaaatgaaataagtggtcggtattcttggaacttccagaaatccaccagttccttcaacttcaccatcacagcctcacaagtcgtggactcagcagtatacttctgtgctctgagtgagtcccataataatgcaggcaacatgctcacctttggagggggaacaaggttaatggtcaaaccc

SEQ ID NO: 43 - DNA, 387 bp, Artificial Sequence
  Synthetic
atgagcatcgggctcctgtgctgtgtggccttttctctcctgtgggcaagtccagtgaatgctggtgtcactcagaccccaaaattccaggtcctgaagacaggacagagcatgacactgcagtgtgcccaggatatgaaccataactccatgtactggtatcgacaagacccaggcatgggactgaggctgatttattactcagcttctgagggtaccactgacaaaggagaagtccccaatggctacaatgtctccagattaaacaaacgggagttctcgctcaggctggagtcggctgctccctcccagacatctgtgtacttctgtgccgcgagccagaacactgaagctttctttggacaaggcaccagactcacagttgta

SEQ ID NO: 44 - protein, 10 aa, Homo sapiens
ILDTAGQEEY

SEQ ID NO: 45 - protein, 27 aa, Artificial Sequence
  Synthetic
RAKRSGSGATNFSLLKQAGDVEENPGP

SEQ ID NO: 46 - protein, 603 aa, Artificial Sequence
  Synthetic
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCAASQNTEAFFGQGTRLTVVEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSESHNNAGNMLTFGGGTRLMVKPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS